(19) United States
(12) Patent Application Publication
(10) Pub. No.: US 2020/0397689 A1
(43) Pub. Date: Dec. 24, 2020

(54) COMPOSITIONS AND METHODS FOR HAIR AND SCALP TREATMENT

(71) Applicant: K STAR DEVELOPMENT K.S.D.S LTD, Jerusalem (IL)

(72) Inventors: Kim SAWDAYEE, Tel Aviv (IL); Elie SAWDAYEE, Tel Aviv (IL)

(21) Appl. No.: 16/940,357

(22) Filed: Jul. 27, 2020

Related U.S. Application Data
(63) Continuation-in-part of application No. PCT/IL2019/050113, filed on Jan. 29, 2019.

(30) Foreign Application Priority Data
Jan. 29, 2018 (IL) 257224

(51) Int. Cl. A61K 8/9789; A61Q 5/02; A61Q 5/00; A61K 36/53

(52) U.S. Cl. CPC A61K 8/9789; A61K 36/53; A61Q 5/002; A61Q 5/02

(57) ABSTRACT

The present invention provides a mixture for the use of synergic treating scalp and hair-disorders, the mixture comprises at least one first compound having premixing specific rotation value, Formula I, peaks within RTs 8.23-8.82 min., at least one second compound, having a Formula II, and RTs 12.67, 12.84, 13.14 and 18.14 min., and at least one third compound having a Formula III and RTs 11.52, 11.59, 15.53, 17.21 and 18.15 min; wherein the mixture is characterized by post mixture specific rotation value Formula IV and by GCMS first, second and third peaks of retention time 11.55, 11.94 and 12.69 min., respectfully; wherein the mixture is characterized by the first peak (11.55 min) to the second peak (11.94 min) ratio between 1:7 to 1:2; the second peak to the third peak (12.69 min.) ratio between 1:4 to 3:2; and the first peak to the third peak ratio of between 1:12 to 1:3 as determined by GCMS peak areas; and further wherein the GCMS is Model GCD 1800, utilized under the following conditions: preparation for analysis was performed by basic extraction (pH 10) in dichloromethane, 3 times, ratio of 1 to 5 sample to solvent; acid extraction (pH −3) in dichloromethane, 3 times, ratio of 1 to 5 sample to solvent; consolidation of the extracts and evaporating in vacuum to a volume of 10 ml; injection of 2 microliters of the sample into the GCMS, using the following program: 60 degrees C. for 7 minutes, increasing at 15 degrees C. per minute to 320 degrees C., staying at 320 degrees C. for 15 minutes.

### CROSS REFERENCE TO RELATED APPLICATIONS

This application is a Continuation-in-Part (CIP) of PCT Patent Application No. PCT/IL2019/050113 having International filing date of Jan. 29, 2019, which claims priority from Israel Patent Application No. 257224 filed on Jan. 29, 2018 entitled COMPOSITIONS AND METHODS FOR HAIR AND SCALP TREATMENT. The contents of the above applications are all incorporated by reference as if fully set forth herein in their entirety.

### FIELD OF THE INVENTION

The current invention generally pertains to method and composition for hair and scalp treatment.

More specifically, the present invention relates to a method for an herbal-based hair and scalp treatment.

### BACKGROUND OF THE INVENTION

Hair from a major part of the external coating of most mammals, however in the human being, hair represents a structure which lost its functional significance during the species evolution process. However, the value of the hair should not be underestimated in emotional and social terms.

Although the hairs do not exhibit a vital biological function, they are of marked importance for appearance, self-esteem and social identity function (Schmitt, Juliano Vilaverde, et al. “Hair loss perception and symptoms of depression in female outpatients attending a general dermatology clinic.” Anais brasileiros de dermatologia 87.3 (2012): 412-41).

Hair is often associated with an individual's identify, and its significance reaches beyond the surface. Hair has psychological, social, and sometimes spiritual meaning. Therefore, loss of hair may have psychological consequences.

Chemically, about 80% of human hair is formed by a protein known as keratin (Kaplin, I. J., A. Schwan, and H. Zahn. “Effects of cosmetic treatments on the ultrastructure of hair.” Cosmet. Toiletries 97.8 (1982): 22-26). Keratin is a laminated complex formed by different structures, which gives the hair strength, flexibility, durability and functionality.

Hair consists of two parts: a shaft and a root, anchored in the follicle, an involution of the epidermis (Hair follicles contain rapidly dividing cells, but the only visible portions are the follicular ostia, through which the hair fibers emerge. Humans are born with a population of approximately five million preformed follicles, only 2 percent of which are on the head. As a human ages, the density of hair decreases.

At the end of the root is a network of blood vessels that supply nutrients to feed the hair and help it grow. This is called the dermal papilla.

In the hair follicle, a dermal papilla (DP), also called follicular papilla, an onion-like structure surrounded by hair bulb matrix), which is a cluster of mesenchymal cells at the base of the follicle, determines the hair thickness, length, and life cycle by adjusting the volume of DP cell aggregate and secretory activity (Jahoda, Colin A B, and Amanda J. Reynolds. “Dermal-Epidermal Interactions-Follicle Derived Cell Populations in the Study of Hair-Growth Mechanisms.” Journal of investigative dermatology 101 (1993). The hair bulb not only produces the shaft, but also provides melanin granules to trichocytes for pigmentation. Surrounding the papilla is bulb. A sebaceous gland, which secretes oily or waxy matter, called sebum, to lubricate and waterproof the skin and hair.

The hair thread has a cylindrical structure, highly organized, formed by inert cells, most of them keratinized and distributed following a very precise and pre-defined design. Hair forms a very rigid structure in the molecular level, which is able to offer the thread both flexibility and mechanical resistance. Hair is considered as a dead mater and it is only alive when it is inserted in the scalp (pilose follicle). When the thread emerges, it becomes dead matter although it appears to be growing since the fiber follows increasing its length by a speed of about 1.0 cm/month (Cade, 1995; Dias, 2004; Longo et al., 2006).

Human hair has about 65-95% of its weight in proteins, more 32% of water, lipid pigments and other components. Chemically, about 80% of human hair is formed by a protein known as keratin (Kaplin, I. J., A. Schwan, and H. Zahn. “Effects of cosmetic treatments on the ultrastructure of hair.” Cosmet. Toiletries 97.8 (1982): 22-26); Wagner, Joekes, 2005) with a high grade of sulfur coming from the amino acid cystine—which is the characteristic to distinguish it from other proteins. Keratin is a laminated complex formed by different structures, which gives the hair strength, flexibility, durability, and functionality.

Hair fiber is composed by three main structures: cuticle, cortex and medulla. The main factor to be considered in the human hair is the high amount of the amino acid cystine, which may be degraded and afterwards may be re-oxidated under a disulphidic bounding form. This is the basis for the permanent curling process. Cystine is very stable; this is the reason why human hair may be found relatively intact, even after several years after the death of an individual (see; Velasco, Maria Valéria Robles, et al. “Hair fiber characteristics and methods to evaluate hair physical and mechanical properties.” Brazilian Journal of pharmaceutical sciences 45.1 (2009): 153-162.).

Hair follicles are complex, highly regenerative, ectodermal-mesodermal structures enriched with stem cells. The hair follicles are highly regenerative, structures enriched with stem cells. The hair follicles lifelong cycles of growth following three distinct stages: anagen (rapid growth), catagen (regression), and telogen (resting period).

The end product of hair follicle proliferation and differentiation is the hair shaft, which, together with its surrounding root sheaths, is derived from epithelial cells. The hair follicle remodels itself during cyclical periods of growth (anagen), regression (catagen), rest (telogen) and shedding (exogen).

Anagen is the growing stage of the hair follicle cycle. Catagen is the regression stage of the hair follicle cycle. During catagen, much of the follicle undergoes programmed cell death (apoptosis), reducing its size as it enters telogen, which is the resting stag of the hair follicle cycle.

Follicular regeneration at the onset of the next anagen phase requires the activation of rarely cycling epithelial stem cells located in the permanent region of the follicle. Stem cell progeny form a new follicle matrix during early anagen, and the hair shaft and inner root sheath are derived from these relatively undifferentiated matrix cells. The size and length of the hair shaft correspond to the size of the hair follicle and to the duration of anagen, respectively. These characteristics vary considerably with body site, and change as a result of disease.

Pigmentation of the hair shaft depends on hair follicle melanocytes, which reside in the hair follicle bulb and deposit melanin into the growing hair shaft. Proliferation of melanocytes occurs during early anagen, and is probably regulated by the factors that control the hair growth cycle

Intrinsic control of each stage involves various factors (e.g., cytokines, hormones, neurotransmitters, and their cognate receptors), (Krause K. Foitzik K. Biology of the hair follicle: the basic. Semin Cutan Med Surg. 2006; 25:2; Stenn K S. Paus R. Controls of hair follicle cycling. Physiol Rev. 2001; 81:449), even though the coordination of these factors remains to be elucidated.

Although the precise role of hair cycling is unclear, it is generally accepted that this process removes debris and parasites from skin surface and encapsulates harmful chemicals within trichocytes (Stenn K. S. Paus R. Controls of hair follicle cycling. Physiol Rev. 2001; 81:449). Hair cycling also serves as a self-protecting system, by removing rapidly proliferating keratinocytes in the catagen phase and preventing oxidative stress and malignant degeneration.

In humans the formation of hair follicles takes place during embryogenesis, and no new hair follicles form after birth. However, the character of individual follicles can change drastically over time. Thicker and darker hairs replace fine lightly pigmented hairs in the beard at puberty. Conversely, thick scalp hairs convert into fine small hairs later in life. Paradoxically, both processes occur in response to the hormone testosterone.

Physical properties of the hair Hair fibers consist of three morphological components namely, the cuticle, the cortex, and the medulla. Layers of cuticle cells form the outer sheath of the hair fiber and are mainly responsible for the cosmetic properties of hair. The cuticle forms a protective barrier for the hair against the outside environment (Robbins C R. Chemical and Physical Behavior of Human Hair. 5th ed. Berlin, Heidelberg: Springer Berlin Heidelberg; 2012). Mechanical properties of the hair are attributed to the cortex, which forms the bulk of the fiber.

Hair mechanical proprieties Hair is very strong. Cortex keratin is responsible for this propriety and its long chains are compressed to form a regular structure which, besides being strong, is flexible (Robbins, C. R. Chemical and physical behavior of human hair. 3.ed. New York: Springer, 1994. 391p). The physical proprieties of hair involve: resistance to stretching, elasticity and hydrophilic power.

Resistance to stretching In general, the weight needed to produce a natural hair thread rupture is 50-100 g. An average head has about 120,000 threads of hair and would support about 12 tons. The resistance to breakage is a function of the diameter of the thread, of the cortex condition, and it is negatively affected by chemical treatments.

Hair elasticity Hair fiber has an elastic characteristic, and it may undergo moderate stretching either wet or dry. Stretching is a hair attribute under the action of a distal force (length) and the thread returns to the original status, when this force stops acting. When dry, the hair thread may stretch 20-30% of its length; and, in contact with water, this may reach up to 50%. In contact with ammonia it becomes more elastic. Chemical and physical treatments, sun exposition and use of electric dryers and heated plates affect this propriety (Juez J. L. and Gimer, L. Ciencia cosmética. 2.ed. Madrid: Soc. Espanhola de Quim. Cosmet., 1983. p.98-119).

Hydrophilic power Hair absorbs water under both liquid and steam form. Keratin may absorb up to 40% of its own weight in water. Hydration is favored by temperature increase, by changing pH and by all the polar solvents which break hydrogen bonds. Hydration changes the fiber elasticity.

Keratin has special affinity for water. This absorption depends on the air relative humidity rate and greatly interferes on all the proprieties of the hair, as: stretching ability, diameter and internal viscosity of the fibers (Juez J. L. and Gimer, L. Ciencia cosmética. 2.ed. Madrid: Soc. Espanhola de Quim. Cosmet., 1983. p. 98-119.; Robbins, C. R. Chemical and physical behavior of human hair. 3.ed. New York: Springer, 1994. 391p).

Surface properties of hair The hair existing in the normal scalp represents a large surface.

Absorption Hair surface retains the thread natural oils (sebum) composed by tensoactive ingredients and some dyers. Absorption of fatty substances is due to a physical process of surface tension. The sebum absorption over the hair occurs by contact with the scalp and transference from a thread to each other.

Friction Friction is the force resisting the movement when a body slides over another one. The cuticle surface has high friction coefficient due to its scale shape and it depends on the cuticle geometry and on the physical-chemical status of the hair. The continuous attrition of a thread over another one damages the cuticle.

From the roots to the extremities the friction coefficient differs in the dry and wet hair thread, and it is enough combing to damage the hair. Several factors influence the friction, such as relative humidity: friction is higher in wet than in dry hair; discoloration of the hair: discoloration increases the friction among threads; permanent waving and straightening: due to the chemical composition and high pH of ingredients the friction is increased (Dias et al., 2005; Dias et al., 2008); shampooing: the more is the detergent power the higher is the friction. The addition of conditioning substances promotes a lower friction coefficient than the one brought by the shampoo, without these substances. Conditioning cream, rinse cream, bath of cream and related products reduce the friction among hair threads (Juez J. L. and Gimer, L. Ciencia cosmética. 2. ed. Madrid: Soc. Espanhola de Quim. Cosmet., 1983. p. 98-119.; Robbins, C. R. Chemical and physical behavior of human hair. 3.ed. New York: Springer, 1994. 391p; ADE, P. Chemical hair straighteners, waves and treatment products. Edison: Croda, 1995. p. 1-21).

Static load When a comb slides over the hair, surface electric load is generated by both friction and high electric resistance of the hair, which makes handling difficult. The static load dispersion is a function of fibers conductivity or electric resistance. Quaternary ammonium salts of long chain increase conductivity on the thread surface and reduce the friction

Shine Shine is one of the most important and desired cosmetic attributes of the hair. From the physical point of view, it is related with the way by which the hair reflects and diffuses the incident light beam.

Thus, any factor which changes light reflection would have influence on shine. Since this is a surface propriety, cuticle is the main responsible by it. Damages on cuticle (opening and breakage of scales), as well as dust particles and scalp secretions built up over threads reduce the shine. The increase of curls bending reduces its visualization and darker hair seems to be more brilliant than the light ones.

When a light beam reaches the hair surface, a part of it is reflected, another part is absorbed, and a third part is dispersed. The amount of light corresponding to each of these phenomena depends on the surface geometry, on the refraction index of the thread, and on the light incidence angle.

Factors influencing the shine perception, by order of importance are: reflection, light dispersion, alignment, and color.

These present favorable effect of hair shine: continuous and thin film over the scales; film with high refraction index; reflection being higher than diffuse dispersion; These present unfavorable effects: higher light dispersion; film coating—irregular or discontinuous—over threads; chemical treatments as permanent waving and discoloration, which cause changes on the flat positioning of the cuticle due to the scales lifting (Reimer, b.; Oldinski, r.; Glover, d. An objective method for evaluating hair shine. Soap, Cosmet., Chem. Spec., Melville, v.10, n. 71, p. 25-32, 1995.; Ishii, m. objective and instrumental methods for evaluation of hair care product efficacy and substantiation claims. in: johnson, d. h., (ed.). Hair and Hair Care. New York: Marcel Dekker, 1997. cap. 10, p. 261-295; Starch, 1999; Schueller, r.; Romanowski, p. Evaluating Shine on Hair. Cosmet. Toiletries, Carol Stream, v.116, n.12, p. 47-52, 2001).

Hair disorders Most hair disorders are not serious, but they are often considered major cosmetic issues that require treatment. Androgenetic alopecia is the most common form of hair loss, although there are a number of other hair conditions that can affect the scalp and other parts of the body.

Hair disorders include hair loss, increased hair growth, and hair structure defects with increased breakage, as well as unacceptable cosmetic appearance, such as reduced shine, strength, curliness, and elasticity. Most common forms of hair loss (alopecia) are caused by aberrant hair follicle cycling and changes in hair follicle morphology.

There are numerous hair disorders that can be diagnosed and evaluated:
a. Androgenetic alopecia is the most common form of hair loss in both men and women. This disorder, also known as male-pattern or female-pattern hair loss, results in permanent hair loss from the scalp. Onset may be as early as puberty: 50% of men affected by age 50, 40% of women by age 70. Male pattern baldness is characterized by thinning of the frontal hairline, bi-temporal recession and hair loss at the crown. Female pattern baldness, is similar to male pattern baldness, except that women can lose their hair in a different pattern than men.
b. Alopecia areata is caused by the immune system attacking hair follicles. As a result, hair falls out in round patches from anywhere on the body. It typically grows back but may fall out again.
c. Traction alopecia describes gradual hair loss that is caused by chronic pulling. Hair that is forced in certain directions, typically through use of braids, ponytails, barrettes, weaves, dreadlocks or protective headgear, can result in significant hair thinning. It is particularly noticeable around the temples and behind the ears.
d. Diffuse alopecia is hair loss from the scalp caused by nutritional deficiencies, imbalance of hormones or other physical or emotional stressors.
e. Chemotherapy-induced alopecia is scalp hair loss that results from chemotherapy, a form of cancer treatment.
f. Telogen effluvium (TE) is the second most common form of hair loss, though little is known about the condition. It is marked by a significant decrease in scalp hair. This condition is related to diffuse alopecia, and diagnosis can be made once other hair disorders have been ruled out. In some extreme cases TE can affect other areas such as the eyebrows or pubic region.
g. Trichotillomania is a psychological condition where a person experiences an impulse to pull their hair out. Hair may be pulled from the scalp, eyebrows or other parts of the body.
h. Lichen planopilaris is a rare inflammatory condition that leads to permanent hair loss, mainly on the scalp. It is usually seen in young adult women though it may also affect men. The cause of this hair disorder is unknown.
i. Frontal fibrosing alopecia is a hair disorder that typically affects women over the age of 50. It causes even hair loss on the front and sides of the scalp, as well as loss of eyebrows. Skin in the affected area usually looks normal but may be pale, shiny or mildly scarred.
j. Central centrifugal cicatricial alopecia is most commonly seen in African Americans. Hair loss begins at the crown of the scalp and progresses outward in a circular pattern. In advanced cases, the damaged hair follicles form scar tissue, preventing future hair growth. There is debate over the cause of this disorder, though use of certain styling agents such as hot hair oil or chemical relaxants are thought to play a role.
k. Folliculitis decalvans can affect hair on any part of the body. Round oval patches of hair loss occur with pustules that surround hair follicles. As hair is shed, follicles are destroyed and scarring results. It affects both men and women and may begin during adolescence or at any in adulthood. The cause is unknown.
l. Dissecting cellulitis is a very rare condition. Pustules develop over the scalp and permanent hair loss results. The disorder can affect anyone though is most commonly seen in African American men (Hillmann, Kathrin, and Ulrike Blume-Peytavi. “Diagnosis of hair disorders.” Seminars in cutaneous medicine and surgery. Vol. 28. No. 1. Frontline Medical Communications, 2009).

Patent document SU1724244 to Gabrielyan concerns cosmetics additives providing a reduction in dandruff and strengthening of hair roots. The object of the invention is the creation of a bioactive anti-dandruff complex to be administered as hair care products in the form of 5% to 10%, providing an optimal reduction of dandruff and a strengthening effect on the roots of hair, which has a positive dermatological effect on the scalp. The composition consists of alcohol-glycerin extracts of wormwood, thousand leaves, horsetail, sea-buckthorn fruits, horseradish root and garlic. Nonetheless, the botanical materials disclosed in this document differ from those of the present invention, and so does the chemical method of preparing the mixture.

Patent document US2006051430 to Arata discloses personal care and home care compositions, including silver dihydrogen citrate. The products are used, for example, in skin care as antiperspirants and deodorants; as personal cleansers; in hair care; as oral care products; and as decorative cosmetics. In addition, these products include antimicrobial agents, which serve to kill or control the growth of undesirable microbes such as bacteria, fungi and viruses. This patent document does not disclose the mixture of plants of the present invention and effects thereof, nor does it relate to the synergistic effects achieved by the combination of plants.

Patent document FR3017300 to Fayou discloses a composition for limiting hair loss and helping hair growth, mainly based on alcohol which is not heated over 60 degrees, and the medicinal plants are individually macerated in specific containers. The three plants include a plant of the Urticaceae family and two plants of the Labiatae family which are Thyme and Rosemary. This patent document does not teach a synergistic effect and the method of preparation of the claimed product is different from that disclosed in the present invention, in which the product is based on infusing plants in boiled water, and not macerating them in ethanol.

Patent document U.S. Pat. No. 6,203,782 to Universal Biolog Inc. discloses methods and compositions for promoting hair growth, preventing or minimizing hair loss, enhancing or restoring hair color or remelanization and treating other hair and skin conditions. The methods include topical application of the compositions to the skin or hair follicles being treated. The compositions include as an essential component a treatment agent in an amount effective for treating the condition and selected from the class of compounds consisting of anole, anethole, analogs thereof, polymers thereof and mixtures thereof. Various combinations of these compounds may be found in herb families including Umbelliferae, Magnoliaceae, Labiatae and Rutaceae. The invention preferably contemplates selecting the treatment agent from the class of herbs consisting of Foeniculum vulgares (fennel seed), Pimpinella anisum (anise), Carum carvi (caraway seeds) and mixtures thereof with each other and/or other herbs. The plants disclosed in this patent document differ from those described in the present application and the preparation time for the therapeutic product disclosed in Patent document U.S. Pat. No. 6,203,782 is significantly longer than that disclosed in the present invention.

Disturbances of hair follicle cycling lie at the heart of most hair growth disorders, and have dramatic effects on visible hair growth and shedding. Despite the psychologic importance of hair preservation, FDA approved therapies for androgenic alopecia is limited to topical minoxidil and oral finasteride. Topical minoxidil solution was developed as a treatment for AGA after its initial oral use for hypertension. Minoxidil increases hair weight through its primary effect of increasing the diameter of existing hairs. Finasteride, 5-a-reductase type II inhibitor, decreases levels of dihydrotestosterone (DHT), which leads to increased hair diameter, growth, and hair counts. Since the approval of finasteride in 1997, there have been no FDA approved medications to treat AGA. The large unmet need for better treatments for hair loss has prompted both physicians and the pharmaceutical industry to develop and pioneer new therapies for hair loss. (Keaney, Terrence. “Emerging Therapies for Androgenetic Alopecia.” Journal of drugs in dermatology: JDD 14.9 (2015): 1036-1040).

### SUMMARY OF THE INVENTION

It is hence one object of the present invention to disclose a synergic water based herbal mixture for the use of treating scalp and hair-disorders comprising at least three herbs, wherein first herb is at least one of Urtica diocia (nettle) or Pipturus albidus (mamaki), having premixing specific rotation value (PSRV) [α]_{D}²⁰=−99 and GCMS 6 peaks within RTs 8.23-8.82 min., second herb is at least one of Thymus vulgaris (thyme) or Salvia officinalis (sage), having a PSRV [α]_{D}²⁰=+33 and RTs 12.67, 12.84, 13.14 and 18.14 min., and third herb is at least one of Rosmarinus officinalis (rosemary) or Mentha viridis (spearmint) having a PSRV [α]_{D}²⁰=−5 and RTs 11.52, 11.59, 15.53, 17.21 and 18.15 min; wherein said mixture is characterized by post mixture specific rotation value [α]_{D}²⁰ =+18 and by GCMS first, second and third peaks of retention time 11.55, 11.94 and 12.69 min., respectively; wherein said mixture is characterized by said first peak (11.55 min) to said second peak (11.94 min) ratio between 1:7 to 1:2; said second peak to said third peak (12.69 min.) ratio between 1:4 to 3:2; and said first peak to said third peak ratio of between 1:12 to 1:3 as determined by GCMS peak areas; and further wherein said GCMS is Model GCD 1800, utilized under the following conditions: preparation for analysis was performed by basic extraction (pH=10) in dichloromethane, 3 times, ratio of 1 to 5 sample to solvent; acid extraction (pH=3) in dichloromethane, 3 times, ratio of 1 to 5 sample to solvent; consolidation of the extracts and evaporating in vacuum to a volume of 10 ml; injection of 2 microliters of the sample into the GCMS, using the following program: 60 degrees C. for 7 minutes, increasing at 15 degrees C. per minute to 320 degrees C., staying at 320 degrees C. for 15 minutes.

It is another object of the present invention to disclose the mixture as defined in any one of the above, wherein first peak area of first peak to second peak to third peak ratio is from 5:17:45 and 5:8:18.

It is another object of the present invention to disclose the mixture as defined in any one of the above, wherein said mixture comprises at least one compound selected from the group consisting of water, ethanol, anti-bacterial agents, moisturizers, hydration agents, penetration agents, preservatives, viscosity-controlling agents anesthetics, vitamins, anti-itch actives, botanical extracts, color agents, enzymes, conditioning agents, darkening or lightening agents, nutrients, micronutrients, fragrances, glitters, hair pigment additives, humectants, mica, minerals, oils, polyphenols, silicones or derivatives thereof, sun-blocks, surfactants, vitamins, waxes, solvents, rheology modifiers, suspending agent, thickeners, hair health actives, anti-dandruff mixtures , anti-oxidants, pigments, abrasives, absorbents, biological actives, buffering agents, chelating agents, opacifying agents, pH adjusters, chelates, perfumes, brighteners, sensates, attractants, dyes, bleaches and any combination thereof.

It is another object of the present invention to disclose the mixture as defined in any one of the above wherein said mixture is provided in a form selected from a group consisting of liquid, solution, capsule, gel, dissolving capsule, capsule comprising a filter, infusion, cream, ointment, electroporation formulation, foam, gel, suspension, emulsion, lotion, microspheres, microbeads, mud, oil, paste, ointment, patch, powder, roller stick, soap, surfactant, spray, shampoo conditioner, a rinse-off conditioner, and any combination thereof.

It is another object of the present invention to disclose the mixture as defined in any one of the above, wherein said mixture is in a form selected from a group consisting of topical, subcutaneous, dermal, oral, and any combination thereof.

It is another object of the present invention to disclose the mixture as defined in any one of the above, wherein said mixture is in a form selected from a tablet, a pill or a granulated powder, nutraceutical, a functional food, a dietary supplement, a food supplement, a pharmaceutical product or a cosmetic product and any combination thereof.

It is another object of the present invention to disclose use the mixture as defined in any one of the above in the preparation of a treatment for hair or scalp disorders.

It is another object of the present invention to disclose use the mixture as defined in any one of the above, wherein said treatment comprises: applying said mixture on said mammalian head, and massaging said mixture in scalp of said mammalian subject.

It is another object of the present invention to disclose use the mixture as defined in any one of the above in the preparation of a treatment for, hair and scalp of a mammalian subject, wherein said mammalian subject is selected from a group consisting of humans, monkeys, cats, cows, dogs, gerbils, goats, guinea pigs, hamsters, horses, mice, prairie dogs, rabbits, rats, sheep and squirrels.

It is another object of the present invention to disclose use the mixture as defined in any one of the above in the preparation of a treatment for hair and scalp of a mammalian subject, wherein said treatment is selected from a group consisting of inducing new hair growth, stimulating new hair growth, increasing hair growth, preventing hair regression, cleansing the hair, cleansing the scalp, beautifying the hair, promoting attraction, altering appearance of hair, and any combination thereof.

It is another object of the present invention to disclose use the mixture as defined in any one of the above in the preparation of a treatment for hair and scalp of a mammalian subject, wherein said mixture is produced by a step of admixing said mixtures with at least one member of a group consisting of water, ethanol, one or more anti-bacterial agents, moisturizers and hydration agents, penetration agents, preservatives, viscosity-controlling agents anesthetics, vitamins, anti-itch actives, botanical extracts, color agents, enzymes, conditioning agents, darkening or lightening agents, fragrances, glitters, hair pigment additives, humectants, mica, minerals, oils, polyphenols, silicones or derivatives thereof, sun-blockers, surfactants, vitamins, micronutrients, nutrients, waxes, solvents, rheology modifiers, suspending agents, thickeners, hair health actives, anti-dandruff actives, anti-oxidants, pigments, abrasives, absorbents, biological actives, buffering agents, chelating agents, opacifying agents, pH adjusters, chelates, perfumes, brighteners, sensates, attractants, dyes, bleaches and any combination thereof.

It is another object of the present invention to disclose use the mixture as defined in any one of the above in the preparation of a treatment for hair and scalp of a mammalian subject, further comprising step of providing said mixture in the form selected from a group consisting of liquid, solution, capsule, gel, dissolving capsule, capsule comprising of filter, infusion, cream, ointment, electroporation formulation, emulsion, foam, gel, suspension, emulsion, lotion, microspheres, microbeads, mud, oil, paste, ointment, patch, powder, roller stick, soap, surfactant, spray, shampoo conditioner, a rinse-off conditioner, and any combination thereof.

It is another object of the present invention to disclose use the mixture as defined in any one of the above in the preparation of a treatment for hair and scalp of a mammalian subject, comprising step of administrating said mixture in a manner selected of a group consisting of topically, subcutaneously, dermally, orally, and any combination thereof.

It is another object of the present invention to disclose use the mixture as defined in any one of the above in the preparation of a treatment for hair and scalp of a mammalian subject, comprising step of administrating said mixture in a manner selected of a group consisting of a sachet a tablet, a pill or a granulated powder, a nutraceutical, a functional food, a dietary supplement, a food supplement, a pharmaceutical product and a cosmetic product.

It is another object of the present invention to disclose use the mixture as defined in any one of the above in the preparation of a treatment for a condition of hair or scalp , wherein said condition is selected from the group consisting of alopecia, accidental injury, damage to hair follicles, surgical trauma, burn wound, radiation or chemotherapy treatment site, incisional wound, donor site wound from skin transplant and ulcer; a desire to modify physical appearance, and any combination thereof; or to enhance one or more hair properties selected from the group consisting of brilliance, fullness, gloss, glow, length, luster, patina, sheen, shine, thickness, volume and any combination thereof.

It is another object of the present invention to disclose a kit for providing a mixture for treating hair or scalp, inducing or stimulating new hair growth, increasing hair growth or preventing hair regression in a mammalian subject, cleansing the hair, cleansing the scalp, beautifying the hair, promoting attraction and altering the appearance of hair and any combination thereof; said kit comprising: a synergic water based herbal mixture comprising at least three herbs, wherein first herb is at least one of Urtica diocia (nettle) or Pipturus albidus (mamaki), having premixing specific rotation value (PSRV) [α]_{D}²⁰ =99 and GCMS 6 peaks within RTs 8.23-8.82 min., second herb is at least one of Thymus vulgaris (thyme) or Salvia officinalis (sage), having a PSRV [α]_{D}²⁰=+33 and RTs 12.67, 12.84, 13.14 and 18.14 min., and third herb is at least one of Rosmarinus officinalis (rosemary) or Mentha viridis (spearmint) having a PSRV [α]_{D}²⁰−5 and RTs 11.52, 11.59, 15.53, 17.21 and 18.15 min; wherein said mixture is characterized by post mixture specific rotation value [α]_{D}²⁰=+18 and by GCMS first, second and third peaks of retention time 11.55, 11.94 and 12.69 min., respectively; wherein said mixture is characterized by said first peak (11.55 min) to said second peak (11.94 min) ratio between 1:7 to 1:2; said second peak to said third peak (12.69 min.) ratio between 1:4 to 3:2; and said first peak to said third peak ratio of between 1:12 to 1:3 as determined by GCMS peak areas; and further wherein said GCMS is Model GCD 1800, utilized under the following conditions: preparation for analysis was performed by basic extraction (pH=10) in dichloromethane, 3 times, ratio of 1 to 5 sample to solvent; acid extraction (pH=3) in dichloromethane, 3 times, ratio of 1 to 5 sample to solvent; consolidation of the extracts and evaporating in vacuum to a volume of 10 ml; injection of 2 microliters of the sample into the GCMS, using the following program: 60 degrees C. for 7 minutes, increasing at 15 degrees C. per minute to 320 degrees C., staying at 320 degrees C. for 15 minutes.

It is another object of the present invention to disclose the kit as defined in any one of the above, wherein peak area of first peak to second peak to third peak ratio is from 5:17:45 to 5:8:18.

It is another object of the present invention to disclose the kit as defined in any one of the above, wherein said application means is selected from the group consisting of, brushes, combs, controlled-release matrices, fabrics, pumps, sprayers, especially aerosol spray dispensers, self-pressurized spray dispensers and non-aerosol spray dispensers, sachets, tea bags, paper tea bags, corn-made tea bags, fabric tea bags, pouches, bags, aluminum bags, cloth bags, plastic bags, filtered bottles, bottles with stainless steel filters, bottles with plastic filters, stainless steel filters, plastic filters, droppers, capsules, balls, compressed cubes, filter, corn paper filters, filter cloths, organic filters, paper filters, ampoules, pouches, flasks, rollers, plastic rollers, plastic rollers and stainless steel, wands and combination thereof.

It is another object of the present invention to disclose the kit as defined in any one of the above, wherein said mixture is provided in a container selected from the group consisting of bottles, cans, tubes, vials, dispensers ,sachets, tea bags, paper tea bags, corn-made tea bags, fabric tea bags, pouches, bags, aluminum bags, cloth bags, plastic bags, filtered bottles, bottles with stainless steel filters, bottles with plastic filters, and any combination thereof.

It is another object of the present invention to disclose the kit as defined in any one of the above, wherein said mixture further comprises at least one member of a group consisting of water, ethanol, anti-bacterial agents, moisturizers and hydration agents, penetration agents, preservatives, viscosity-controlling agents anesthetics, vitamins, anti-itch actives, botanical extracts, color agents, enzymes, conditioning agents, darkening or lightening agents, nutrient, micronutrient, fragrance, glitter, hair pigment additives, humectants, mica, minerals, oils, polyphenols, silicones or derivatives thereof, sun-blocks, surfactants, vitamins, waxes, solvents, rheology modifiers, suspending agent, thickeners, hair health actives, anti-dandruff actives, anti-oxidants, pigments, abrasives, absorbents, biological actives, buffering agents, chelating agents, opacifying agents, pH adjusters, chelates, perfumes, brighteners, sensates, attractants, dyes, bleaches and any combination thereof.

It is another object of the present invention to disclose the kit as defined in any one of the above, wherein said mixture is provided in the form selected from a group consisting of liquid, solution, capsule, gel, dissolving capsule, capsule comprising of filter, infusion, cream, ointment, electroporation formulation, emulsion, foam, gel, suspension, emulsion, lotion, microspheres, microbeads, mud, oil, paste, ointment, patch, powder, roller stick, soap, surfactant, spray, shampoo conditioner, a rinse-off conditioner, and any combination thereof.

It is another object of the present invention to disclose the kit as defined in any one of the above, wherein said mixture is configured to be administrable in a manner selected from a group consisting of topical, subcutaneous, dermal, oral, tablet, a pill or a granulated powder, nutraceutical, a functional food, a dietary supplement, a food supplement, a pharmaceutical product or a cosmetic product and any combination thereof.

It is another object of the present invention to disclose the kit as defined in any one of the above, wherein said mammalian subject has a condition a of hair or scalp selected from the group consisting of alopecia, accidental injury, damage to hair follicles, surgical trauma, burn wound, radiation or chemotherapy treatment site, incisional wound, donor site wound from skin transplant and ulcer; a desire to modify physical appearance, or any combination thereof.

It is another object of the present invention to disclose the kit as defined in any one of the above, wherein said mixture configured to enhance one or more hair properties selected from the group consisting of brilliance, fullness, gloss, glow, length, luster, patina, sheen, shine, thickness, volume and any combination thereof.

It is one object of the present invention to disclose a method for preparation of a synergic water based herbal mixture for scalp or hair treatment, wherein said method comprises steps of: mixing at least three herbs, wherein first herb is at least one of Urtica diocia (nettle) or Pipturus albidus (mamaki), having premixing specific rotation value (PSRV) [α]_{D}²⁰=−99 and GCMS 6 peaks within RTs 8.23-8.82 min., second herb is at least one of Thymus vulgaris (thyme) or Salvia officinalis (sage), having a PSRV [α]_{D}²⁰=+33 and RTs 12.67, 12.84, 13.14 and 18.14 min., and third herb is at least one of Rosmarinus officinalis (rosemary) or Mentha viridis (spearmint), having a PSRV [α]_{D}²⁰=−5 and RTs 11.52, 11.59, 15.53, 17.21 and 18.15 min.; thereby producing a mixture; and immersing said mixture in boiling water for 10 to 180 minutes; wherein said mixture is characterized by post mixture specific rotation value [α]_{D}²⁰=+18 and by GCMS first, second and third peaks of retention time 11.55, 11.94 and 12.69 min., wherein said mixture is characterized by said first peak (11.55 min) to said second peak (11.94 min) ratio between 1:7 to 1:2; said second peak to said third peak (12.69 min.) ratio between 1:4 to 3:2; and said first peak to said third peak ratio of between 1:12 to 1:3 as determined by GC peak areas; and further wherein utilizing GCMS, Model GCD 1800 under the following conditions: preparation for analysis was performed by basic extraction (pH=10) in dichloromethane, 3 times, ratio of 1 to 5 sample to solvent; acid extraction (pH=3) in dichloromethane, 3 times, ratio of 1 to 5 sample to solvent; consolidation of the extracts and evaporating in vacuum to a volume of 10 ml; injection of 2 microliters of the sample into the GCMS, using the following program: 60 degrees C. for 7 minutes, increasing at 15 C per minute to 320 degrees C., staying at 320 degrees C. for 15 minutes.

It is another object of the present invention to disclose the method as defined in any one of the above, wherein said step of immersing is followed by boiling said mixture and then cooking the boiled mixture for additional up to 60 minutes in 40 ° C. to 80° C.

It is one object of the present invention to disclose a cosmetic product comprising an effective measure of a mixture of at least three herbs, wherein first herb is at least one of Urtica diocia (nettle) or Pipturus albidus (mamaki) having premixing specific rotation value (PSRV) [α]_{D}²⁰=99 and GCMS 6 peaks within RTs 8.23-8.82 min, second herb is at least one of Thymus vulgaris (thyme) or Salvia officinalis (sage) having a PSRV [α]_{D}²⁰=+33 and RTs 12.67, 12.84, 13.14 and 18.14 min., and third herb is at least one of Rosmarinus officinalis (rosemary) or Mentha viridis (spearmint) having a PSRV [α]_{D}²⁰=−5 and RTs 11.52, 11.59, 15.53, 17.21 and 18.15 min; thereby producing a mixture of herbs, adding said mixture of herbs to boiling water; boiling until said mixture said mixture is characterized by post mixture specific rotation value [α]_{D}²⁰=+18 and by GCMS first, second and third peaks of retention time 11.55, 11.94 and 12.69 min., wherein said mixture is characterized by said first peak (11.55 min) to said second peak (11.94 min) ratio between 1:7 to 1:2; said second peak to said third peak (12.69 min.) ratio between 1:4 to 3:2; and said first peak to said third peak ratio of between 1:12 to 1:3 as determined by GC peak areas; and further wherein utilizing GCMS, Model GCD 1800 under the following conditions: preparation for analysis was performed by basic extraction (pH=10) in dichloromethane, 3 times, ratio of 1 to 5 sample to solvent; acid extraction (pH=3) in dichloromethane, 3 times, ratio of 1 to 5 sample to solvent; consolidation of the extracts and evaporating in vacuum to a volume of 10 ml; injection of 2 microliters of the sample into the GCMS, using the following program: 60 C for 7 minutes, increasing at 15 C per minute to 320 C, staying at 320 C for 15 minutes.

### BRIEF DESCRIPTION OF THE FIGURES

The accompanying drawings, which are included to provide a further understanding of the invention and are incorporated in and constitute a part of this specification, illustrate embodiments of the invention and together with the description serve to explain the principles of the invention.

FIG. 1 Photos of a female (Female #1), treated with the composition of the current invention. Photos (A) were taken 5 hours before shampooing compared to photos taken 5 hours following 18 times of shampooing (B), of the same female individual. Photos were taken in a back view (A1, B1), a side view (A2, B2), and from an overhead view (A3, B3);

FIG. 2a Micrographs of hair of Female #1, taken under a light microscope at a 25-fold magnification. Hairs A and B were taken from an individual before use of the composition, while hairs C and D were taken following treatment of said composition for about a month. The micrographs were taken by Kaps Som 62 dental microscope with Sony Next camera connected by TV tube.

FIG. 2b Micrographs of hair of Female #1, taken under a light microscope at a 100-fold magnification. Hairs A and B were taken from an individual before use of the composition, while hairs C and D were taken following treatment of said composition for 18 times of shampooing, about a month.

The micrographs were taken by light microscope Olympus B.H-2, connected to an Olympus camera, using DP70 software.

FIG. 2c Micrographs of hair of Female #1, taken under a light microscope at a 200-fold magnification. Hairs A and B were taken from an individual before use of the composition, while hairs C and D were taken following treatment of said composition 18 times of shampooing, about a month.

The micrographs were taken by light microscope Olympus B.H-2, connected to an Olympus camera, using DP70 software.

FIG. 2d Micrographs of hair of Female #1, taken under a light microscope at a 400-fold magnification. Hairs A and B were taken from an individual before use of the composition, while hairs C and D were taken following treatment of said composition 18 times of shampooing, about a month.

The micrographs were taken by light microscope Olympus B.H-2, connected to an Olympus camera, using DP70 software.

FIG. 3 Micrographs of hair of Female #2, taken under a light microscope at a 100-fold (A), a 200-fold (B), a 400-fold (C) magnification. Hairs A and B and C were taken from an individual following treatment of said composition for about seven months.

The micrographs were taken by light microscope Olympus B.H-2, connected to an Olympus camera, using DP70 software.

FIG. 4 A GC-MS chromatogram of the mixture according to one embodiment of the invention.

FIGS. 5A and 5B A GC-MS chromatogram of the first compound according to one embodiment of the invention.

FIG. 6 A GC-MS chromatogram of the second compound according to one embodiment of the invention.

FIG. 7 A GC-MS chromatogram of the third compound according to one embodiment of the invention.

FIG. 8A-FIG. 8C A kit according to one to one embodiment of the invention.

FIG. 9 A map of hair location on the scalp.

FIG. 10 An electron microscope micrograph of hair the vertex area, of Female #3.

FIG. 11 An electron microscope micrograph of hair the rear area, of Female #3.

FIG. 12 An electron microscope micrograph of hair the front area, of Female #3.

FIG. 13 An electron microscope micrograph of hair the parietal area, of Female #3.

FIG. 14 An electron microscope micrograph of hair the nape area, of Female #3.

FIG. 15 An electron microscope micrograph of hair the temporal area, of Female #3.

### DETAILED DESCRIPTION OF THE PREFERRED EMBODIMENTS

Reference will now be made in detail to the preferred embodiments of the present invention, examples of which are illustrated in the accompanying drawings. The current invention discloses, in a non-limiting manner, a hair and scalp treating, water based herbal mixture for the use of synergic treating scalp and hair-disorders (hereinafter “mixture”) which comprises, inter alia, at least one first herb having premixing specific rotation value (PSRV) [α]_{D}²⁰=−99 and GCMS 6 peaks within RTs 8.23-8.82 min., at least one second herb, having a PSRV [α]_{D}²⁰=+33 and RTs 12.67, 12.84, 13.14 and 18.14 min., and at least one third herb having a PSRV [α]_{D}²⁰=−5 and RTs 11.52, 11.59, 15.53, 17.21 and 18.15 min; wherein said mixture is characterized by post mixture specific rotation value [α]_{D}²⁰=+18 and by GCMS first, second and third peaks of retention time 11.55, 11.94 and 12.69 min., respectively; wherein said mixture is characterized by said first peak (11.55 min) to said second peak (11.94 min) ratio between 1:7 to 1:2; said second peak to said third peak (12.69 min.) ratio between 1:4 to 3:2; and said first peak to said third peak ratio of between 1:12 to 1:3 as determined by GCMS peak areas; and further wherein said GCMS is Model GCD 1800, utilized under the following conditions: preparation for analysis was performed by basic extraction (pH=10) in dichloromethane, 3 times, ratio of 1 to 5 sample to solvent; acid extraction (pH=3) in dichloromethane, 3 times, ratio of 1 to 5 sample to solvent; consolidation of the extracts and evaporating in vacuum to a volume of 10 ml; injection of 2 microliters of the sample into the GCMS, using the following program: 60 degrees C. for 7 minutes, increasing at 15 degrees C. per minute to 320 degrees C., staying at 320 degrees C. for 15 minutes.

The term “about” refers to any value being up to 20% lower or greater the defined measure. GC conditions are defined below.

The current invention also discloses aforesaid hair and scalp treating mixture, wherein peaks area of Group A to Group B to Group C ratio is about (3-7):(10-22):(30-60) in general, and more specifically, about 5:17:45.

It is in the scope of the invention wherein materials of the first compound (GROUP A) are one or more materials selected from a group consisting of “2-Borneol” or “Bingpian” or “(1R,2S,4R)-rel-1,7,7-TriMethylbicyclo[2.2.1]heptan-2-ol” or “Bornel (crystal)” or “Endo-1,7,7-trimethyl-bicyclo[2.2.1]heptan-2-ol 2-Camphanol” or “1,7,7-Trimethylbicyclo(2.2.1)heptan-2-ol” or “endo-; 1,7,7-trimethyl-endo-Bicyclo[2.2.1]heptan-2-ol” or “2-Bornanol, endo-” or “507-70-0” OR “(1S-endo)-1,7,7-Trimethylbicyclo(2.2.1)heptan-2-ol” Or “000464-45-9” or “(−)-Borneol” or “1-Bornyl alcohol; Borneol, (1S,2R,4S)-(−)-; 1-2-Bornanol” or “1-2-Camphanol; 1-Borneol; L-Borneol” or “Linderol” or “NGAI camphor” or “Bicyclo(2.2.1)heptan-2-ol, 1,7,7-trimethyl-, (1S,2R,4S)-” OR “1192-62-7” or “2-Acetylfuran” or “1-(2-furanyl)ethanone” or “Furan, 2-acetyl-; acetylfuran; Ethanone, 1-(2-furanyl)-” or “2-Furyl methyl ketone”; “2-Acetylfurane” or “1-(2-furanyl)ethanone (acetylfuran)” or “(2-furanyl)-1-ethanone” or “1-(2-Furanyl)-ethanone (2-acetylfuran)” or “furyl methyl ketone” or “1-(2-Furyl)ethanone” or “Ketone, 2-furyl methyl; Methyl 2-furyl ketone”.

It is also in the scope of the invention wherein materials of the second compound (GROUP B) are one or more materials selected from a group consisting of “2-PINEN-4-ONE” or “4,6,6-TRIMETHYLBICYCLO[3.1.1]HEPT-3-EN-2-ONE” or “VERBENONE” or “4,6,6-trimethyl-bicyclo[3.1.1]hept-3-en-2-on” or Berbenone or “2-Pinen-4-one, 4,6,6-” or “Trimethylbicyclo[3.1.1]hept-3-en-2-one” or “2,6,6-Trimethylbicyclo[3.1.1]hepta-2-ene-4-one” or “Pina-2-ene-4-one” or “80-57-9” OR “503-93-5” OR “Eucarvone” OR “2,6,6-Trimethyl-2,4-cycloheptadien-1-one” OR “2,4-cycloheptadien-1-one, 2,6,6-trimethyl-” OR “2,6,6-trimethyl-2,4-cycloheptadien-1-one” OR “ 2,6,6-trimethylcyclohepta-2,4-dien-1-one” OR “2,4-cycloheptadien-1-one-2,6,6-trimethyl” OR “73657-70-2” OR “2-Pyridinecarbonitrile,1,2,5,6-tetrahydro-1,3,4-trimethyl-” OR “NSC 375118” OR “2-Pyridinecarbonitrile, 1,2,5,6-tetrahydro-1,3,4-trimethyl-” or “1,4,5-trimethyl-3,6-dihydro-2H-pyridine-6-carbonitrile” OR “1,4,5-trimethyl-3,6-dihydro-2H-pyridine-6-carbonitrile”.

It is yet also in the scope of the invention wherein materials of the third compound (GROUP C) are one or more materials selected from a group consisting of “1-Hydroxy-2-methyl-5-isopropylbenzene” or “2-Hydroxy-1-methyl-4-(1-methylethyl)benzene; 2-hydroxy-4-(2-propyl)toluene” or “2-hydroxy-p-cymen” or “2-methyl-5-(1-methylethyl)-pheno” or “2-para-cymenol; 2-p-Cymenol” or “3-isopropyl-6-methyl-pheno” or Carvacrol Or “499-75-2” OR “26967-65-7” OR “Phenol, diethyl-” OR “ Diethylphenol” OR “2,3-diethylphenol; 2,5-diethylphenol; 3,5-diethylphenol ” “Phenol, diethyl-” OR “Diethylphenol” OR “89-83-8” OR “Thymol” OR “p-Cymen-3-ol” OR “2-Isopropyl-5-methylphenol; m-Thymol” OR “2-Isopropyl-5-methylphenol (thymol); Isopropyl cresol” OR “5-Methyl-2-(1-methylethyl)-phenol” OR “Phenol, 5-methyl-2-(1-methylethyl)-” OR “Thyme camphor” OR “3-Hydroxy-p-cymene” OR “3 -Methyl-6-isopropyl phenol” OR “5-Methyl-2-isopropyl phenol” OR “6-Isopropyl-m-cresol” OR “6-Isopropyl-3-methylphenol” OR “m-Cresol, 6-isopropyl-” OR “p-Cymene, 3-hydroxy-” OR “Phenol, 2-isopropyl-5-methyl-” OR “Thymic acid” OR “1-Hydroxy-5-methyl-2-isopropylbenzene” OR “1-Methyl-3-hydroxy-4-isopropylbenzene” OR “3-p-Cymenol” OR “3-Hydroxy-1-methyl-4-isopropylbenzene” OR “5-Methyl-2-isopropyl-1-phenol” OR “Isopropyl-m-cresol” OR “2-Hydroxy-1-isopropyl-4-methylbenzene” OR “NSC 11215”.

The present invention also discloses hair and scalp treating mixtures as defined in any of the above, wherein the mixture further comprises at least one member of a group consisting, inter alia, water, water-miscible alcohol such as ethanol, anti-bacterial agents, moisturizers and hydration agents, penetration agents, preservatives, viscosity-controlling agents anesthetics, vitamins, anti-itch actives, botanical extracts, color agents, enzymes, conditioning agents, darkening or lightening agents, nutrient, micronutrient, fragrance, glitter, hair pigment additives, humectants, mica, minerals, oils, polyphenols, silicones or derivatives thereof, sunblocks, surfactants, vitamins, waxes, solvents, rheology modifiers, suspending agent, thickeners, hair health actives, anti-dandruff actives, anti-oxidants, pigments, abrasives, absorbents, biological actives, buffering agents, chelating agents, opacifying agents, pH adjusters, chelates, perfumes, brighteners, sensates, attractants, dyes, bleaches and any combination thereof.

The present invention also discloses hair and scalp treating mixtures as defined in any one of the above, wherein the mixture comprises at least one preservative, inter alia a preservative named Geogard Ultra™, comprising gluconolactone, calcium gluconate, sodium benzoate and sodium bicarbonate.

The present invention also discloses hair and scalp treating mixtures as defined in any of the above, wherein the mixture is provided in a form selected from a group consisting, inter alia, liquid, solution, capsule, gel, dissolving capsule, capsule comprising of filter, infusion, cream, ointment, electroporation formulation, emulsion, foam, gel, suspension, emulsion, lotion, microspheres, microbeads, mud, oil, paste, ointment, patch, powder, roller stick, soap, surfactant, spray, shampoo conditioner, a rinse-off conditioner, and any combination thereof.

The present invention also discloses hair and scalp treating mixtures as defined in any of the above, wherein the mixture is configured to be administrable in a manner selected from a group consisting, inter alia, a group comprising topical, subcutaneous, dermal, oral, tablet, a pill or a granulated powder, nutraceutical, a functional food, a dietary supplement, a food supplement, a pharmaceutical product or a cosmetic product and any combination thereof.

The present invention also discloses a method for treating hair and scalp of a mammalian subject. The method comprises, inter alia, steps of: obtaining a mixture comprising at least one compound selected from Group A having a GC retention time 11.55 min.; at least one compound selected from Group B having a GC retention time of 11.94 min.; and at least one compound selected from Group C having a GC retention time of 12.69 min.; wherein GC peak area of Group A to Group B ratio is ranging from about 1:7 to about 1:2; peak area of Group B to Group C ratio is ranging from about 1:4 to about 3:2; and peak area of Group A to Group C ratio is ranging from about 1:12 to 1:3. It is according to a specific embodiment of the invention wherein peaks area of Group A to Group B to Group C ratio is about (3-7):(10-22):(30-60) in general, and more specifically, about 5:17:45.

It is in the scope of the invention wherein aforesaid method further comprising step(s) selected from step (a) applying said mixture on said mammalian head, and step (b) massaging the mixture in scalp of the mammalian subject.

The term “mammalian” refers hereinafter to any member of a group consisting of humans, including males, females, toddlers and grown up persons, and other mammals such as monkeys, pets e.g., cats, cows, dogs, gerbils, goats, guinea pigs, hamsters, farm animals, e.g., horses, mice, prairie dogs, rabbits, rats, sheep, squirrels and a combination thereof.

It is in the scope of the invention wherein the method as defined above is provided useful for a treatment selected from a group consisting, inter alia, inducing and/or stimulating new hair growth, increasing hair growth or preventing hair regression, cleansing the hair, cleansing the scalp, beautifying the hair promoting attraction, altering the appearance of hair and any combination thereof.

It is also in the scope of the invention wherein the method as defined in any of the above is provided useful for producing a mixture by a step or set of steps of admixing mixtures with at least one material or diluent selected from a group consisting, inter alia, water, water miscible alcohol such as ethanol, one or more anti-bacterial agents, moisturizers and hydration agents, penetration agents, preservatives, viscosity-controlling agents anesthetics, vitamins, anti-itch actives, botanical extracts, color agents, enzymes, conditioning agents, darkening or lightening agents, fragrance, glitter, hair pigment additives, humectants, mica, minerals, oils, polyphenols, silicones or derivatives thereof, sun-blockers, surfactants, vitamins, micronutrients, nutrients, waxes, solvents, rheology modifiers, suspending agent, thickeners, hair health actives, anti-dandruff actives, anti-oxidants, pigments, abrasives, absorbents, biological actives, buffering agents, chelating agents, opacifying agents, pH adjusters, chelates, perfumes, brighteners, sensates, attractants, dyes, bleaches and any combination thereof.

It is also in the scope of the invention wherein the method as defined in any of the above comprises step(s) of providing said mixture in the form selected from a group consisting, inter alia, liquid, solution, capsule, gel, dissolving capsule, capsule comprising of filter, infusion, cream, ointment, electroporation formulation, emulsion, foam, gel, suspension, emulsion, lotion, microspheres, microbeads, mud, oil, paste, ointment, patch, powder, roller stick, soap, surfactant, spray, shampoo conditioner, a rinse-off conditioner, and any combination thereof.

It is also in the scope of the invention wherein the method as defined in any of the above comprising step(s) of administrating the mixture in a manner selected of a group consisting, inter alia, topically, subcutaneously, dermally, orally, and any combination thereof.

It is also in the scope of the invention wherein the method as defined in any of the above comprising step(s) of administrating said mixture in a manner selected of a group consisting of a tablet, a pill or a granulated powder, a nutraceutical, a functional food, a dietary supplement, a food supplement, a pharmaceutical product and a cosmetic product.

It is also in the scope of the invention wherein the method as defined in any of the above is provided useful for treating a condition selected from the group consisting, inter alia, alopecia, accidental injury, damage to hair follicles, surgical trauma, burn wound, radiation or chemotherapy treatment site, incisional wound, donor site wound from skin transplant and ulcer; a desire to modify physical appearance, and any combination thereof; or to enhance one or more hair properties selected from the group consisting of brilliance, fullness, gloss, glow, length, luster, patina, sheen, shine, thickness, volume and any combination thereof.

The invention further discloses a kit for either inducing or stimulating new hair growth, increasing hair growth or preventing hair regression in a mammalian subject, cleansing the hair, cleansing the scalp, beautifying the hair, promoting attraction and altering the appearance of hair. This kit comprises, inter alia, a mixture comprising at least one compound selected from Group A having a GC retention time 11.55 min.; at least one compound selected from Group B having a GC retention time of 11.94 min.; and at least one compound selected from Group C having a GC retention time of 12.69 min.; wherein GC peak area of Group A to Group B ratio is ranging from about 1:7 to about 1:2; peak area of Group B to Group C ratio is ranging from about 1:4 to about 3:2; and peak area of Group A to Group C ratio is ranging from about 1:12 to 1:3. It is according to a specific embodiment of the invention wherein peaks area of Group A to Group B to Group C ratio of the mixture in the kit is about (3-7):(10-22):(30-60) in general, and more specifically, about 5:17:45.

It is in the scope of the invention wherein the aforesaid kit comprises a mixture which is contained in a vessel. The vessel is selected from the group consisting, inter alia, containers, bottles, brushes, cans, combs, controlled-release matrices, fabrics, pumps, sprayers, especially aerosol spray dispensers, self-pressurized spray dispensers and non-aerosol spray dispensers, tubes, vials, a tea bag, a paper tea bad, a corn-made tea bag, a fabric tea bag, a dye, a pouch, a bag, an aluminum bag, a cloth bag, a plastic bag, a bottle, a filtered bottle, a bottle with stainless steel filter, a bottle with a plastic filter, a stainless steel filter, a plastic filter, a dropper, capsules, balls, compressed cubes, a filter, a corn paper filter, a filter cloth, an organic filter, a paper filter, an ampoule, a pouch, a flask, a roller, a plastic roller, a flask, a plastic roller and stainless steel, wands and combination thereof.

It is also in the scope of the invention wherein the kit as defined in any of the above comprising one or more mixtures, at least one mixture comprises or diluted with at least one member of a group consisting, inter alia, water, water-miscible alcohol such as ethanol, anti-bacterial agents, moisturizers and hydration agents, penetration agents, preservatives, viscosity-controlling agents anesthetics, vitamins, anti-itch actives, botanical extracts, color agents, enzymes, conditioning agents, darkening or lightening agents, nutrient, micronutrient, fragrance, glitter, hair pigment additives, humectants, mica, minerals, oils, polyphenols, silicones or derivatives thereof, sun-blocks, surfactants, vitamins, waxes, solvents, rheology modifiers, suspending agent, thickeners, hair health actives, anti-dandruff actives, anti-oxidants, pigments, abrasives, absorbents, biological actives, buffering agents, chelating agents, opacifying agents, pH adjusters, chelates, perfumes, brighteners, sensates, attractants, dyes, bleaches and any combination thereof.

It is also in the scope of the invention wherein the kit as defined in any of the above comprising a mixture which is provided in a form selected from a group consisting, inter alia, liquid, solution, capsule, gel, dissolving capsule, capsule comprising of filter, infusion, cream, ointment, electroporation formulation, emulsion, foam, gel, suspension, emulsion, lotion, microspheres, microbeads, mud, oil, paste, ointment, patch, powder, roller stick, soap, surfactant, spray, shampoo conditioner, a rinse-off conditioner, and any combination thereof.

It is also in the scope of the invention wherein the kit as defined in any of the above comprising a mixture which is configured to be administrable in a manner selected from a group consisting, inter alia, a group comprising topical, subcutaneous, dermal, oral, tablet, a pill or a granulated powder, nutraceutical, a functional food, a dietary supplement, a food supplement, a pharmaceutical product or a cosmetic product and any combination thereof.

It is also in the scope of the invention wherein the kit as defined in any of the above comprising a mixture configured to treat a mammalian subject which has a condition or conditions. The condition to be treated are selected from the group consisting, inter alia, of alopecia, accidental injury, damage to hair follicles, surgical trauma, burn wound, radiation or chemotherapy treatment site, incisional wound, donor site wound from skin transplant and ulcer; a desire to modify physical appearance, or any combination thereof.

It is also in the scope of the invention wherein the kit as defined in any of the above comprising a mixture configured to enhance one or more hair properties selected from the group consisting of brilliance, fullness, gloss, glow, length, luster, patina, sheen, shine, thickness, volume and any combination thereof.

According to one embodiment of the invention, mixture(s) of the current invention is/are a mixture of herbs, prepared as either infusion (following soaking in boiled water), a cooked product-after cooking in medium temperate, vapors or being provided as an extract, using either organic or inorganic solvents.

The term “herbs” refers hereinafter to one or more herbs, spices, plants, parts, mixtures, fresh, dry, intact used as is, chopped, milled, powder or extracts thereof. It is yet also in the scope of the invention wherein materials of GROUP D are one or more herbs selected from a group consisting of at least three herbs: the first herb is a member of the Urticaceae family, preferably first herb is at least one of Urtica diocia (nettle) or Pipturus albidus (mamaki); the second herb is a member of the Nepetoideae family, preferably second herb is at least one of Thymus vulgaris (thyme) or Salvia officinalis (sage); and the third herb is a member of the Lamiaceae family, preferably third herb is at least one of Rosmarinus officinalis (rosemary) or Mentha viridis (spearmint).

### EXAMPLE 1

In one example, at least three of different herbs selected from Group D were mixed. The mixture included 46% of at least one first herb, 27% of at least one second herb and 27% of at least one third herb. Said mixture then boiled in 200 ml deionized water. The obtained extract mixture was analyzed: at least one compound selected from Group A having a GC retention time 11.55 min.; at least one compound selected from Group B having a GC retention time of 11.94 min.; and at least one compound selected from Group C having a GC retention time of 12.69 min.; wherein GC peak area of Group A to Group B ratio is ranging from about 1:7 to about 1:2; peak area of Group B to Group C ratio is ranging from about 1:4 to about 3:2. Peaks area of Group A to Group C ratio was ranging from about 1:12 to 1:3. The filtered extract, allowed to cool to room temperature, was introduced on scalps of various groups of female's and male's for about one hour. One group utilized aforesaid mixture in a liquid solution; Second group utilized aforesaid mixture in a gel form; third group utilized aforesaid mixture in a capsulated manner; forth group utilized aforesaid mixture in cream-like ointment; fifth group utilized aforesaid mixture in a water in oil emulsion; sixth group utilized aforesaid mixture as a foam; seventh group utilized aforesaid mixture in oil-miscible solution; eighth group utilized aforesaid mixture as a milled powder; ninth group utilized aforesaid mixture as (i) soap, (ii) shampoo, and (iii) conditioner. Hair condition (including alopecia's scoring) in those nine test groups was significantly better then controlled group that utilized two-herb based mixtures.

### EXAMPLE 2

Preparation of an Infusion

No-rinse treatment The herbal mixture (about 10 g) was boiled in 350 ml water, with or without a filter. After about 90 minutes, liquid cools down and reaches room temperature. The color of the water turns dark brown. The cooled mixture was poured on wet hair (in the bath) after the overlap, both on the scalp and on the hair itself including hair ends. Scalp was massaged slightly and was not washed. The mixture is recommended to use after each hair shampooing, at least 3 times a week. The hair can be dried with a hairdryer.

Preparation of a Cooked Product

A mixture of the three different herbs selected from Group D (about 10 g, total weight) was used. GC peaks area of Group A to Group B to Group C ratio was about 5:17:45. The said mixture then boiled in either a tea-bag, filtered or in a non-filtered state, in 350 ml of water. When the water reaches a boiling temperature, lower the temperature and further heat (about 60° C.) another about 10 minutes. Wait for it to cool down. Filtration is possible (if filter was not used during cooking).

Preparation of a Water-Washable Product

The three different herbs selected from Group D were provided in a mixture (about 10 g) and boiled in 350 ml water. The obtained extract was analyzed by means of GCMS and revealed 4 peaks, at RT=11.55, RT=11.94, RT=12.69 and RT=12.84. The peak areas measured were as follows: RT=11.55, area=5.25, height=25 mm; RT=11.94, area=17.18, height=41 mm; RT=12.69, area=44.89, height=90 mm; and RT=12.84, area=32.69, height=78 mm. The extract was then incorporated in both solvent-based and waterborne cosmetic preparations comprising, inter alia, one or more members of a group consisting of a water-miscible alcohol, such as ethanol, anti-bacterial agents, moisturizers and hydration agents, penetration agents, preservatives, viscosity-controlling agents anesthetics, vitamins, anti-itch actives, botanical extracts, color agents, enzymes, conditioning agents, darkening or lightening agents, nutrient, micronutrient, fragrance, glitter, hair pigment additives, humectants, mica, minerals, oils, polyphenols, silicones or derivatives thereof, sun-blocks, surfactants, vitamins, waxes, solvents, rheology modifiers, suspending agent, thickeners, hair health actives, anti-dandruff actives, anti-oxidants, pigments, abrasives, absorbents, biological actives, buffering agents, chelating agents, opacifying agents, pH adjusters, chelates, perfumes, brighteners, sensates, attractants, dyes, bleaches and any combination thereof.

Two Methods for Preparation of a Mixture Useful for Treating Scalp and Hair

First method comprising two steps: First step is admixing an effective measure of at least one first herb selected from Group D, an effective measure of at least one second herb selected from Group D, and an effective measure of at least one third herb selected from Group D; said mixture, when boiled, is characterized by GC retention time where at least one compound selected from Group A having a GC retention time 11.55 min.; at least one compound selected from Group B having a GC retention time of 11.94 min.; and at least one compound selected from Group C having a GC retention time of 12.69 min.; wherein GC peak area of Group A to Group B ratio is ranging from about 1:7 to about 1:2; peak area of Group B to Group C ratio is ranging from about 1:4 to about 3:2; and peak area of Group A to Group C ratio is ranging from about 1:12 to 1:3; and then:

Second step, immersing said mixture in boiling water for about 10 to about 180 minutes.

This method was provided useful, inter alia, for treating hair disorders, alopecia, accidental injury, damage to hair follicles, surgical trauma, burn wound, radiation or chemotherapy treatment site, incisional wound, donor site wound from skin transplant and ulcer.

Additionally or alternatively, the second method comprising three steps. Frist step of admixing an effective measure of at least one first herb selected from Group D, an effective measure of at least one second herb selected from Group D, and an effective measure of at least one third herb selected from Group D; said mixture, when boiled and cooked, is characterized by GC retention time where at least one compound selected from Group A having a GC retention time 11.55 min.; at least one compound selected from Group B having a GC retention time of 11.94 min.; and at least one compound selected from Group C having a GC retention time of 12.69 min.; wherein GC peak area of Group A to Group B ratio is ranging from about 1:7 to about 1:2; peak area of Group B to Group C ratio is ranging from about 1:4 to about 3:2; and peak area of Group A to Group C ratio is ranging from about 1:12 to 1:3.

Second step of immersing said mixture of in boiling water for about 10 to about 180 minutes; and boiling said mixture; and then

Third step of cooking the boiled mixture for additional up to 60 minutes in about 40° C. to about 80° C.

This method was provided useful for, inter alia, providing the treated hair disorders including improving brilliance, fullness, gloss, glow, length, luster, patina, sheen, shine, thickness, and volume.

### EXAMPLE 3

Questionnaires and Results

Two cosmetic products were tested. The first cosmetic product comprises an effective measure of at least one first herb selected from Group D, an effective measure of at least one second herb selected from Group D, and an effective measure of at least one third herb selected from Group D; said mixture, when boiled, is characterized by GC retention time where at least one compound selected from Group A having a GC retention time 11.55 min.; at least one compound selected from Group B having a GC retention time of 11.94 min.; and at least one compound selected from Group C having a GC retention time of 12.69 min.; wherein GC peak area of Group A to Group B ratio is ranging from about 1:7 to about 1:2; peak area of Group B to Group C ratio is ranging from about 1:4 to about 3:2; and peak area of Group A to Group C ratio is ranging from about 1:12 to 1:3; and then immersing said mixture of (b) in boiling water for about 10 to about 180 minutes. The second cosmetic product comprises an effective measure of at least one compound selected from Group A having a GC retention time 11.55 min.; at least one compound selected from Group B having a GC retention time of 11.94 min.; and at least one compound selected from Group C having a GC retention time of 12.69 min.; wherein GC peak area of Group A to Group B ratio is ranging from about 1:7 to about 1:2; peak area of Group B to Group C ratio is ranging from about 1:4 to about 3:2; and peak area of Group A to Group C ratio is ranging from about 1:12 to 1:3; and then immersing said mixture of (b) in boiling water for about 10 to about 180 minutes.

A study was performed on 40 women who used the mixture defined above for about two weeks, overall about 18 times the mixture was used for each woman. The results of the questionnaires were very similar. They all answered the questionnaire after 10 times the product was used. The effects were already apparent after a small number of uses. All of them were satisfied with the mixture of the current invention. All the treated human subjects indicated a change in their hair condition, whether they had a specific problem or not. The mixture of the current invention treats a diseased hair, but also with healthy hair but thin, etc.

According to the detailed questionnaire of 10^{th} time treatment and the longer examination (18^{th} time treatment) of one female, the significant improvement in hair loss and overall hair appearance are evident after 5 washings (shampooing), but significant results can be seen after longer use. Once the treatment stops, the hair slowly returns to normal in terms of its symptoms and appearance.

The questionnaires included the aforementioned queries: Are the instructions for use clear enough? Is the use with the product simple or complicated? Can you characterize the hair smell following use? Did you feel any change in your hair following use with the product? How many treatments are needed for causing the change? What is the main contribution of the product, if any in the following characteristics: Oily hair, hair loss, thin hair, hair volume, feeling fuller hair? Would you like to use the product again? If the product is marketed—what will be your weekly use? Would you like to use a daily dosage or a weekly dosage? Would you recommend other to use this product? How would you define the product? Any other comments/remarks? The results of the questionnaires are summarized in Table 1 and Table 2 below:

TABLE 1
The effect of the 10^{th} time use mixture, according to the above questionnaires.
The questionnaires were summarized and evaluated using various characteristics:

| | |
|---|---|
| Shining | a shiner hair, a sense of “sparkling” hair, glamor. |
| Volume | a feeling that the hair is more swollen in the positive sense of the word. |
| | Instead of flat hair and close to the scalp and face, hair has more |
| | presence in terms of volume as a result of repeated use of the product. |
| Feeling fuller hair | there is a feeling that the hair is full in terms of the amount of hair |
| | on the head. Feeling that there is more hair on your head. |
| A feeling of thickening of the hair | there is a feeling that the hair itself is thicker, a feeling that the washing |
| | of the plants wraps the hair follicle and creates a kind of “cover”. |
| Hair is stronger | hair feels stronger, less broken, edges less split. |
| | The hair fiber is durable and stronger. |
| Healthy hair | there is a feeling that the hair is no longer broken easily, does not tear |
| | easily. The root is more durable and stronger. The hair itself is more |
| | durable. The new hair that grows also feels stronger and healthier. |
| Scalp and hair are less oily | scalp is much less oily than it was. A feeling of a clean scalp, both of the |
| | scales and of the fat the skin produces. You can wash less often a week |
| | and feel that the hair still looks good and still clean, without an oily |
| | appearance. Women who used to shampoo every day - while using the product |
| | dropped and 3-4 overlaps per week! Every other day. This is significant and |
| | facilitates the routine of life and cultivation much more. |
| Reducing hair loss | less hair is falling out. Hairs fall much less during the day when the hand on |
| | the hair and without any contact with the hair. We examined women with |
| | dramatic dropout problems and women with light hair loss. All reported |
| | significant improvement. |
| Smell | the product has a pleasant smell that lingers over time, a natural smell. |
| The product nourishes any hair type | (curly, smooth, corrugated, etc.) |

TABLE 2
The effect of the 18^{th} time use mixture, according to
the above questionnaires:
The effect of 18^{th} time treatment was as follows:

The hair is brighter, the hair feels more alive, more pleasant and soft, the
scales are gone, the scalp is no longer as greasy as it was, the hair is not
oily, the hair feels stronger and fuller.
I have thin, volume-less hair, the use of the brew makes my hair become
much more volume.
The more product users see these changes. Initial change can already be
seen between first and third use. A significant change is already
seen in the fifth use.
In terms of hair loss, after 18 rinses with the product, when I move
my hand in my hair I see there are far fewer hairs falling out.
The smell of the product is very pleasant.

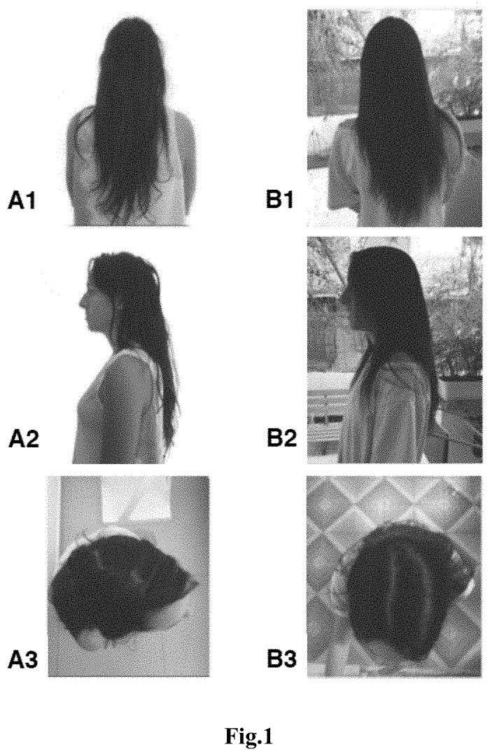

Reference is now made to FIG. 1 that depicts a female (Female #1), treated with the mixture of the current invention. Photos (A) were taken 5 hours before shampooing compared to photos taken 5 hours following 18 times of shampooing (B), of the same female individual. Photos were taken in a back view (A1, B1), a side view (A2, B2), and from an overhead view (A3, B3);

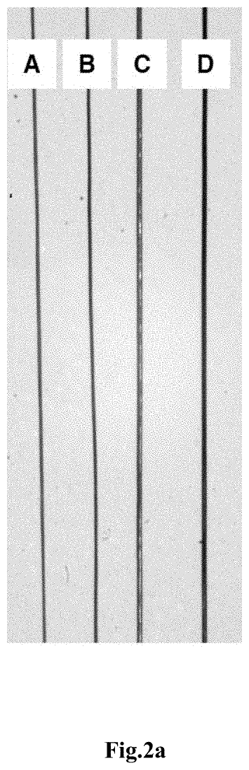

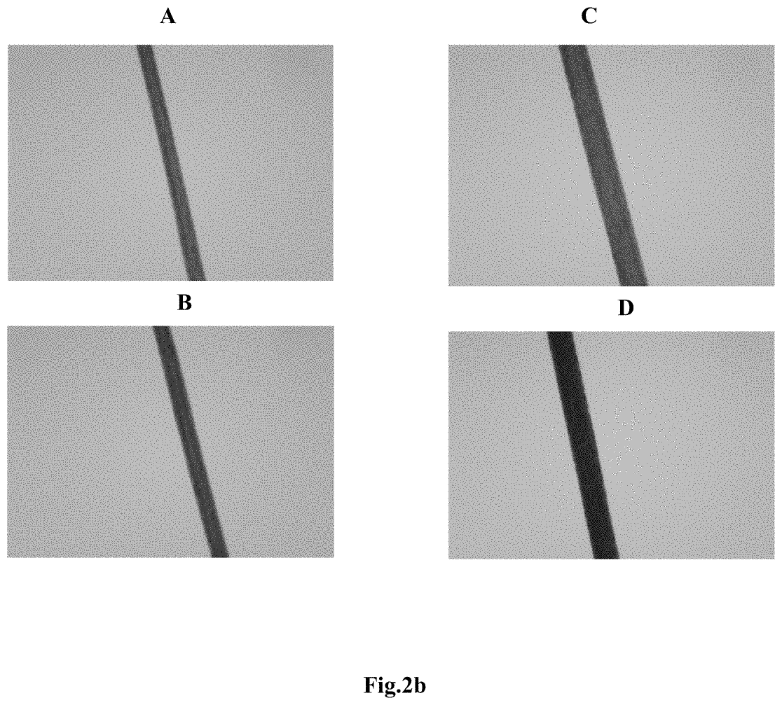

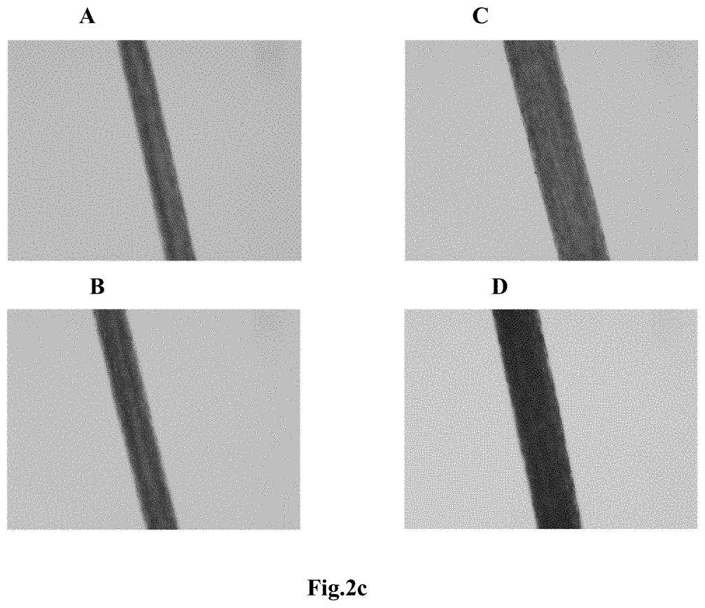

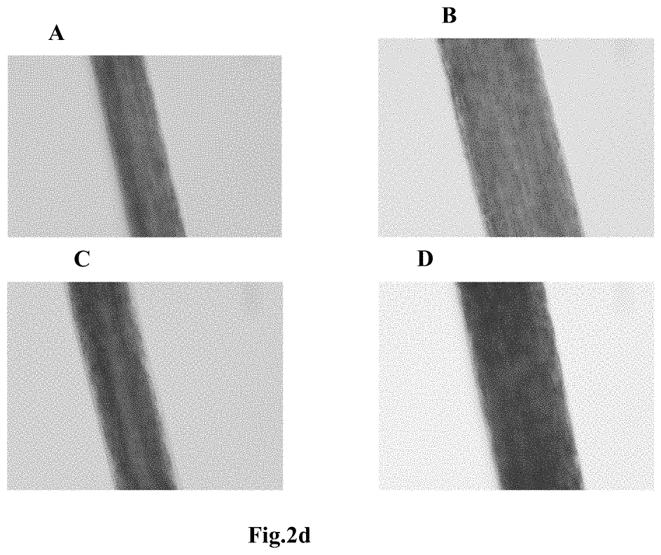

Reference is now made to FIG. 2a that depicts micrographs of hair of Female #1, taken under a light microscope at a 25-fold magnification. Hairs A and B were taken from an individual before use of the mixture, while hairs C and D were taken following treatment of said mixture for about a month. The micrographs were taken by Kaps Som 62 dental microscope with Sony Next camera connected by TV tube.

Reference is now made to FIG. 2b that depicts Micrographs of hair of Female #1, taken under a light microscope at a 100-fold magnification. Hairs A and B were taken from an individual before use of the mixture, while hairs C and D were taken following treatment of said mixture for 18 times of shampooing, about a month.

The micrographs were taken by light microscope Olympus B.H-2, connected to an Olympus camera, using DP70 software.

Reference is now made to FIG. 2c that depicts micrographs of hair of Female #1, taken under a light microscope at a 200-fold magnification. Hairs A and B were taken from an individual before use of the mixture, while hairs C and D were taken following treatment of said mixture 18 times of shampooing, about a month.

The micrographs were taken by light microscope Olympus B.H-2, connected to an Olympus camera, using DP70 software.

Reference is now made to FIG. 2d that depicts micrographs of hair of Female #1, taken under a light microscope at a 400-fold magnification. Hairs A and B were taken from an individual before use of the mixture, while hairs C and D were taken following treatment of said mixture 18 times of shampooing, about a month.

The micrographs were taken by light microscope Olympus B.H-2, connected to an Olympus camera, using DP70 software.

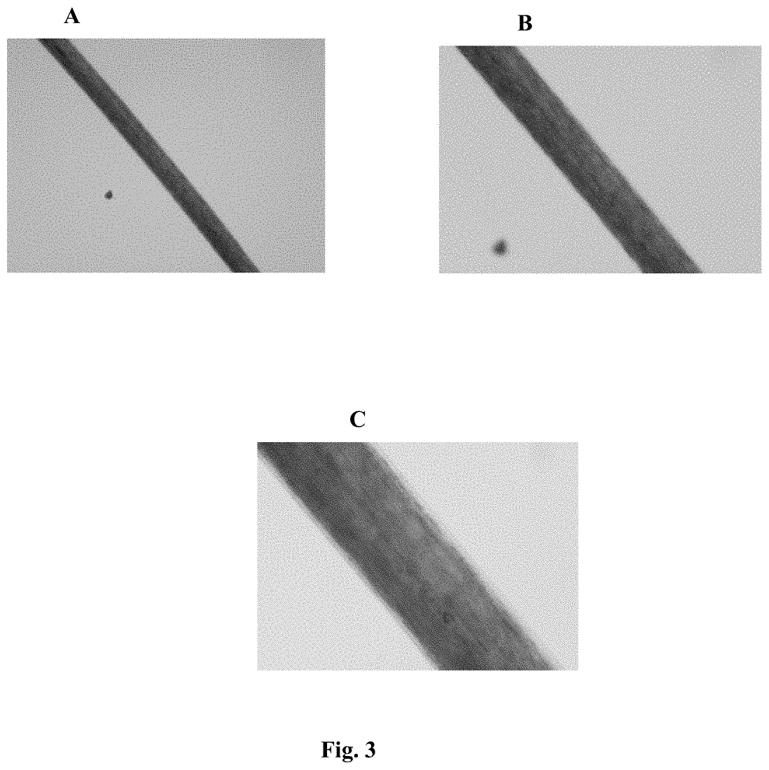

Reference is now made to FIG. 3 that depicts Micrographs of hair of Female #2, taken under a light microscope at a 100-fold (A), a 200-fold (B), a 400-fold (C) magnification. Hairs A and B and C were taken from an individual following treatment of said mixture for about seven months.

The micrographs were taken by light microscope Olympus B.H-2, connected to an Olympus camera, using DP70 software.

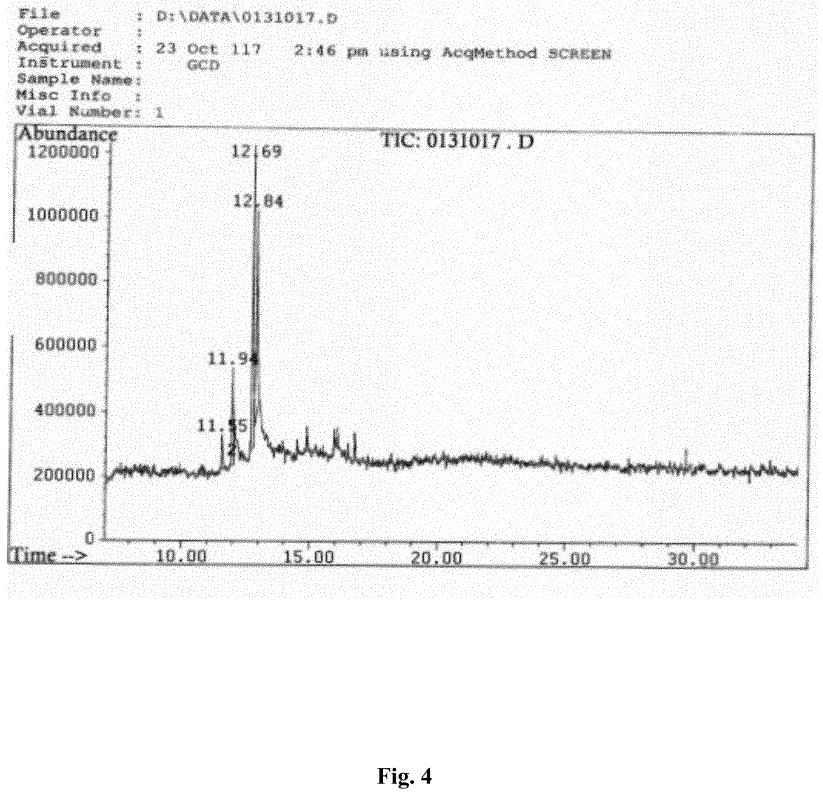

Reference is now made to FIG. 4 depicting A GC-MS chromatogram of the mixture according to one embodiment of the invention

### EXAMPLE 4

GC parameters The infusion mixture of the current invention has been analyzed by Gas Chromatograph, GCMS, Model GCD 1800. Preparation for analysis was performed by Basic extraction (pH=10) in dichloromate, 3 times, ratio of 1 to 5 sample to solvent Acid extraction (pH=3) in dichloromethane, 3 times, ratio of 1 to 5 sample to solvent. Consolidation of the extracts and evaporating in vacuum to a volume of 10 ml. Injection of 2 microliters of the sample into the GCMS, using the following program: 60 C for 7 minutes, increasing at 15 C per minute to 320 C, staying at 320 C for 15 minutes.

GC parameters The infusion mixture of the current invention has been analyzed by Gas Chromatograph, GCMS, Model GCD 1800. Preparation for analysis was performed by Basic extraction (pH=10) in dichloromate, 3 times, ratio of 1 to 5 sample to solvent Acid extraction (pH=3) in dichloromethane, 3 times, ratio of 1 to 5 sample to solvent. Consolidation of the extracts and evaporating in vacuum to a volume of 10 ml. Injection of 2 microliters of the sample into the GCMS, using the following program: 60 C for 7 minutes, increasing at 15 C per minute to 320 C, staying at 320 C for 15 minutes.

Reference is now made to FIG. 4-7 depicting A GC-MS chromatogram of the mixture according to one embodiment of the invention

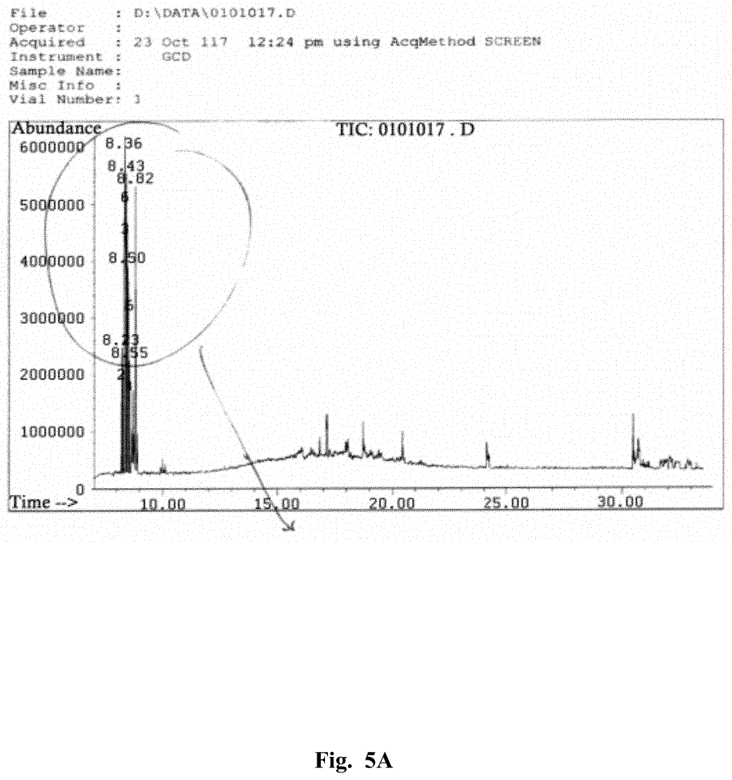

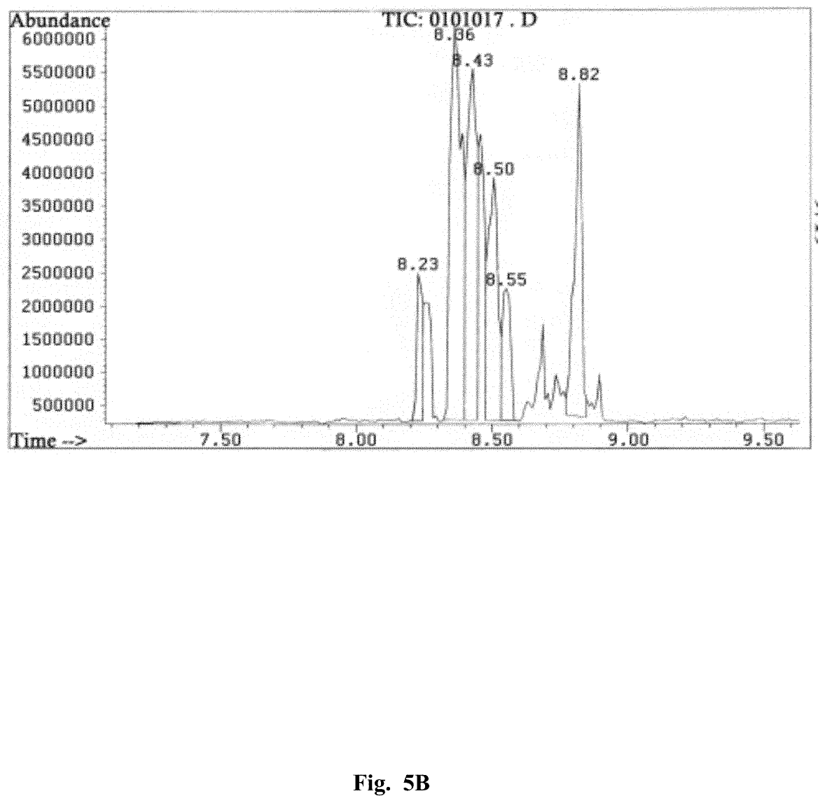

The GCMS Chromatogram, for the analyzed compounds of the first herb revealed 6 peaks, at RT=8.23, RT=8.36, RT=8.43, RT=8.50, RT=8.55 and RT=8.82. The peak areas measured were as follows: RT=8.43, area=6.39; RT=8.36, area=32.40; RT=8.43, area=22.85; RT=8.50, area=15.49; RT=8.55, area=6.63, and RT=8.82, area=16.27. GC chromatogram is depicted in FIGS. 5A and 5B.

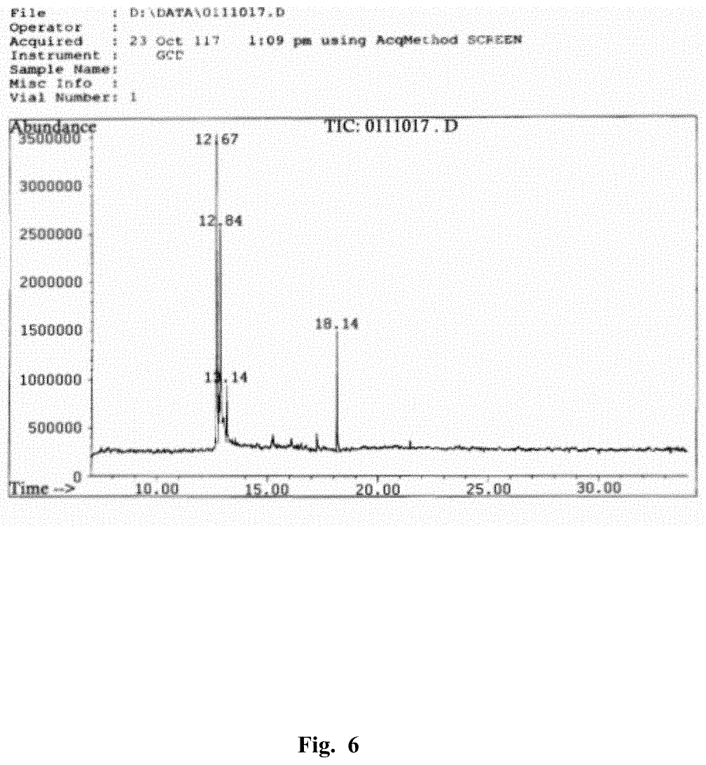

The GCMS Chromatogram, for the analyzed compounds of the second herb revealed 4 peaks, at RT=12.67, RT=12.84, RT=13.14, and RT=18.14. The peak areas measured were as follows: RT=12.67, area=52.22; RT=12.84, area=27.21; RT=13.14, area=7.90; RT=18.14, and area=12.67. GC chromatogram is depicted in FIG. 6.

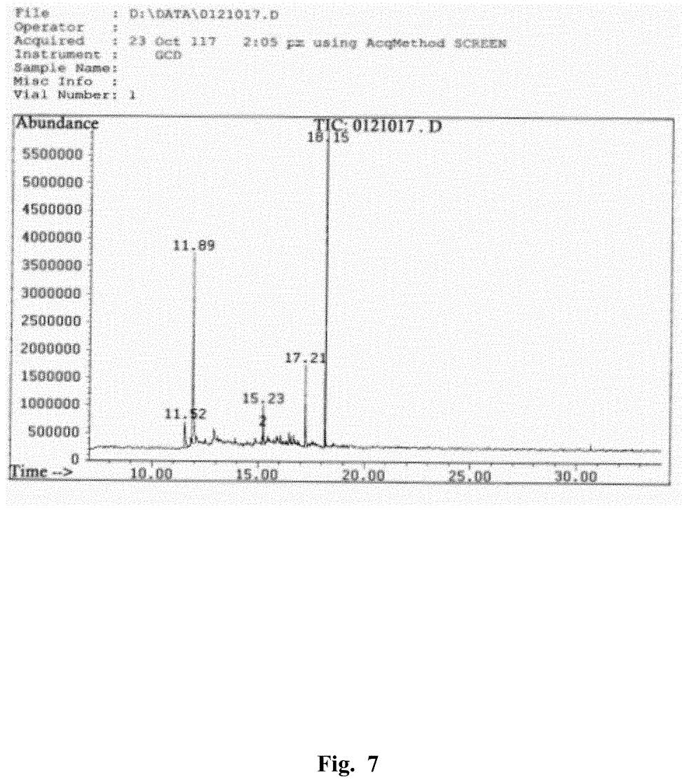

The GCMS Chromatogram, for the analyzed compounds of the third herb revealed 5 peaks, at RT=11.52, RT=11.89, RT=15.23, RT=17.21 and RT=18.15. The peak areas measured were as follows: RT=11.52, area=8.73; RT=11.89, area=37.95; RT=15.23, area=7.04; RT=17.21, area=12.32; RT=18.15, and area=33.96. GC chromatogram is depicted in FIG. 7.

The GCMS Chromatogram, analyzed following preparation of the infusion of the current invention (the mixture), revealed 4 peaks, at RT=11.55, RT=11.94, RT=12.69 and RT=12.84. The peak areas measured were as follows: RT=11.55, area=5.25, height=25 mm; RT=11.94, area=17.18, height =41 mm; RT=12.69, area=44.89, height=90 mm; and RT=12.84, area=32.69, height=78 mm. GC chromatogram is depicted in FIG. 4.

Having thus described a preferred embodiment, it should be apparent to those skilled in the art that certain advantages of the described method and apparatus have been achieved. It should also be appreciated that various modifications, adaptations, and alternative embodiments thereof may be made within the scope and spirit of the present invention. The invention is further defined by the following claims.

### EXAMPLE 5

Chirality: Chirality test, for measuring the specific rotation values of the compounds and the mixture of the invention was performed using Autopol automatic polarimeter by Rudolph research analytical.

The measurements of the three compounds and their mixture reveled the following values:
a. The first herb has a premixing specific rotation value of [α]_{D}²⁰=99;
b. The second herb has a premixing specific rotation value of [α]_{D}²⁰=+33;
c. The third herb has a premixing specific rotation value of [α]_{D}²⁰=−5; and
d. The mixture of herbs has a post mixture specific rotation value of [α]_{D}²⁰=+18.

In a preferred embodiment first herb is Urtica dioica (nettle) or Pipturus albidus (mamaki), second herb is Thymus vulgaris (thyme) or Salvia officinalis (sage) and third herb is Rosmarinus officinalis (rosemary) or Mentha viridis (spearmint).

### EXAMPLE 6

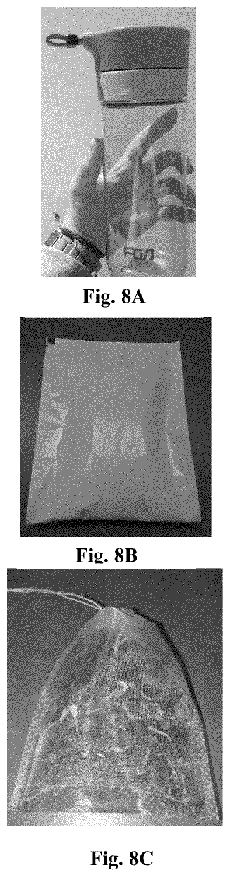

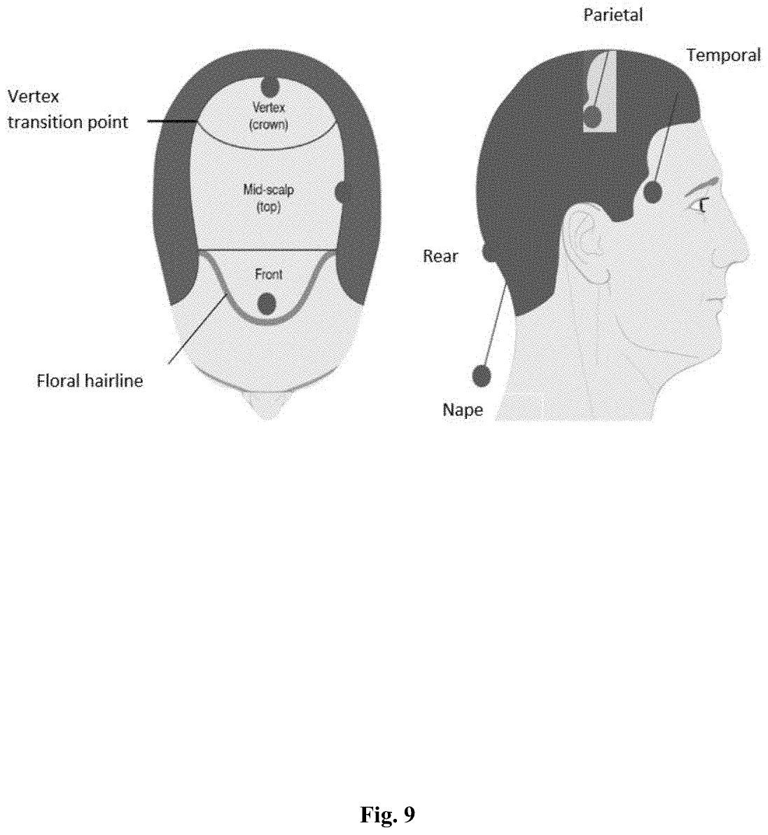

A kit and use instructions: A preferred embodiment discloses the kit for treatment of scalp and hair is packed and contains: a clear bottle containing an infuser (FIG. 8A), and sealed sachets (FIG. 8B), each of the sachets includes a “tea bag” made of organza (or nylon). The “tea bag” includes about 7 grams of the mixture (FIG. 8C); see technical specification of the bag in Table 3.

Directions for use: boil tap water; fill up the bottle (FIG. 8A) up to 200 ml with boiled water; open one sachet and take out the “tea bag”; insert the contents of the “tea bag” into the boiled water in the bottle; mix thoroughly, wait for 10 minutes, add tap water up to 500 ml; check if temperature is suitable for skin treatment; and then pour on scalp and hair (after the hair was shampooed and rinsed); dry with a towel; leave on scalp and hair until next shampooing.

Recommended frequency for use: at least twice a week, preferably three times a week.

### EXAMPLE 7

The term leachable refers herein after being capable of being removed from a substance by a percolating liquid.

The term Miscibility/refers herein after to the property of two substances to mix in all proportions (that is, to fully dissolve in each other at any concentration), forming a homogeneous solution.

A preferred embodiment discloses a synergic water based herbal mixture for the use of treating scalp and hair-disorders, comprising at least three herbs, wherein the first herb is a member of the Urticaceae family, preferably first herb is at least one of Urtica diocia (nettle) or Pipturus albidus (mamaki), having premixing specific rotation value (PSRV) [α]_{D}²⁰=−99 and GCMS 6 peaks within RTs 8.23-8.82 min.; the second herb is a member of the Nepetoideae family, preferably second herb is at least one of Thymus vulgaris (thyme) or Salvia officinalis (sage), having a PSRV=+33 and RTs 12.67, 12.84, 13.14 and 18.14 min.; and the third herb is a member of the Lamiaceae family, preferably third herb is at least one of Rosmarinus officinalis (rosemary) or Mentha viridis (spearmint), having a PSRV-5 and RTs 11.52, 11.59, 15.53, 17.21 and 18.15 min; wherein said mixture is characterized by post mixture specific rotation value and by GCMS first, second and third peaks of retention time 11.55, 11.94 and 12.69 min., respectively; wherein said mixture is characterized by said first peak (11.55 min) to said second peak (11.94 min) ratio between 1:7 to 1:2; said second peak to said third peak (12.69 min.) ratio between 1:4 to 3:2; and said first peak to said third peak ratio of between 1:12 to 1:3 as determined by GCMS peak areas; and further wherein said GCMS is Model GCD 1800, utilized under the following conditions: preparation for analysis was performed by basic extraction (pH=10) in dichloromethane, 3 times, ratio of 1 to 5 sample to solvent; acid extraction (pH=3) in dichloromethane, 3 times, ratio of 1 to 5 sample to solvent; consolidation of the extracts and evaporating in vacuum to a volume of 10 ml; injection of 2 microliters of the sample into the GCMS, using the following program: 60 degrees C. for 7 minutes, increasing at 15 degrees C. per minute to 320 degrees C., staying at 320 degrees C. for 15 minutes.

### EXAMPLE 8

Electron Microscopy Analysis

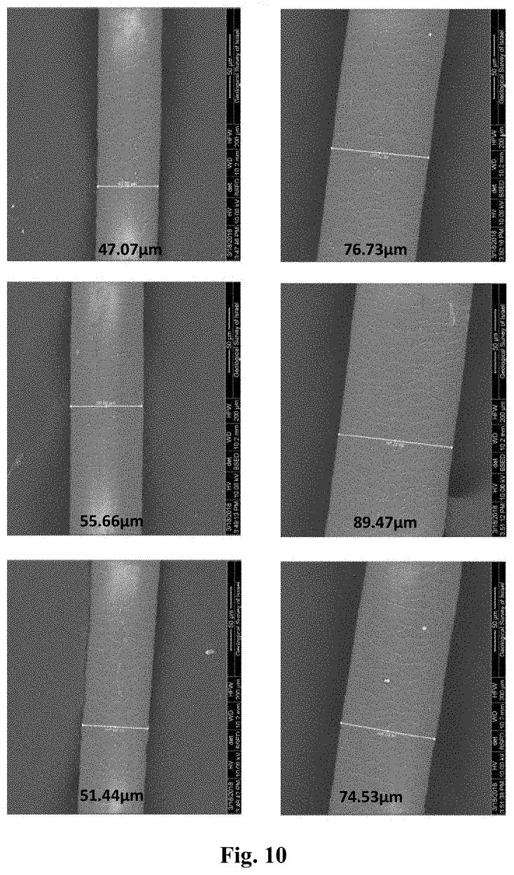

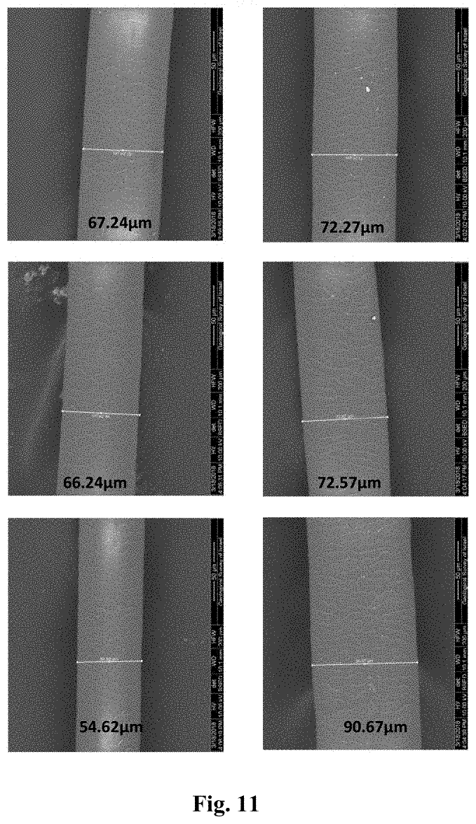

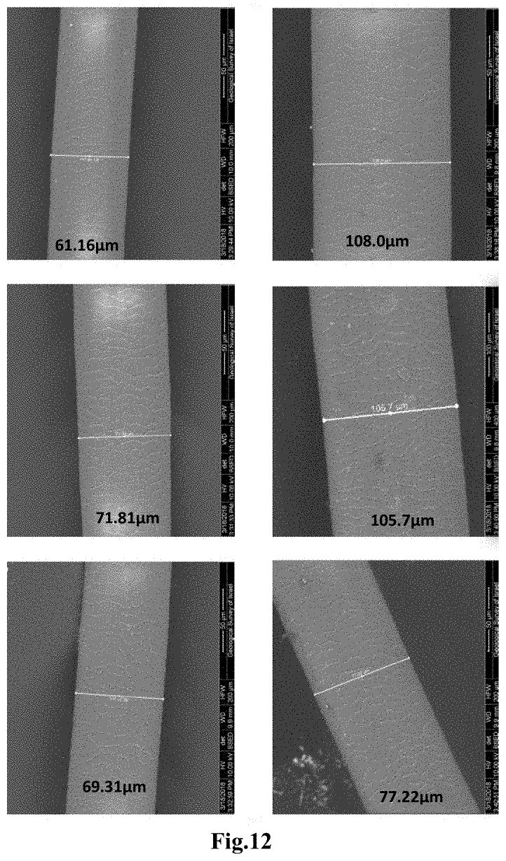

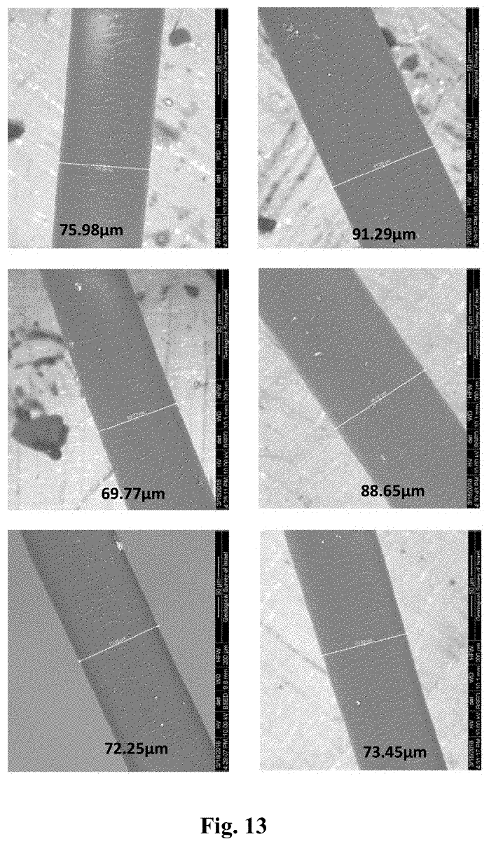

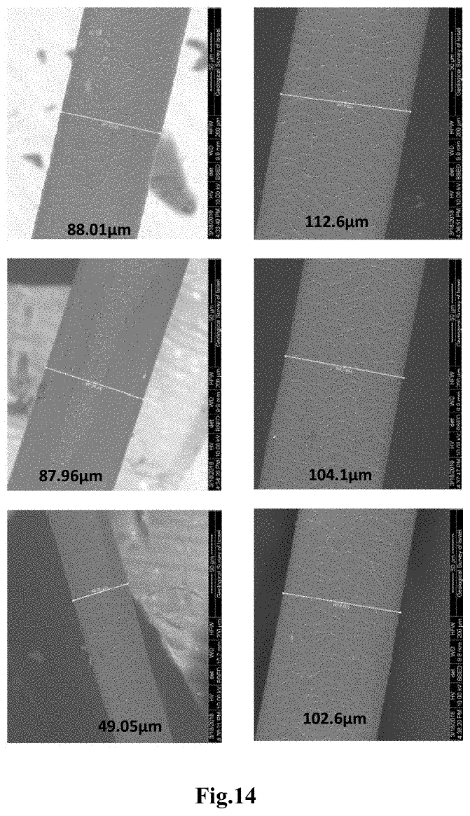

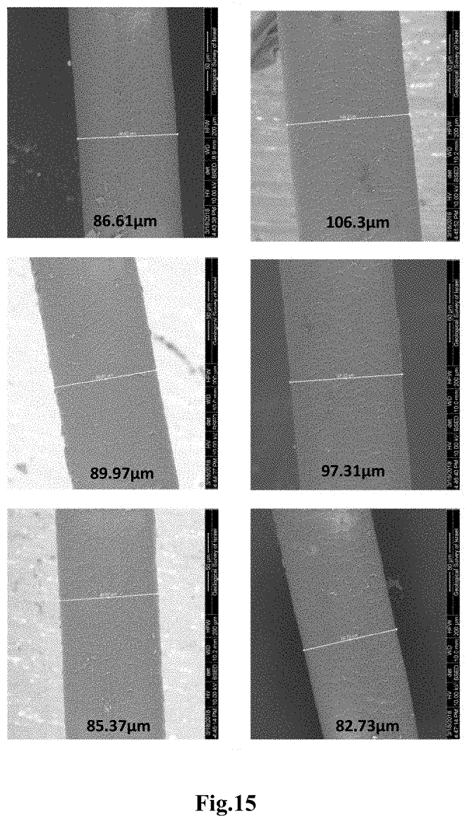

The experimenter, female #3, is a female, age 24, who has thin, falling and oily hair; and also suffers of hair thinning. The experimenter was treated for a month, at least twice a week, and was photographed in two time points during the treatment period:
a. Before the treatment—the scalp was photographed at various directions. Hairs where taken from 6 different locations on the scalp (n=3 hairs for each location; see FIGS. 10-15).
b. After using the mixture for about a month, hairs were taken from the same 6 locations on the scalp and were compared by the electronic microscope analysis.

The results show a significant (P value=5.47E-07, Student's T-test,), thickening of the hair after use of a month (see Table 4)

TABLE 4
Hair width before and after treatment (as determined by Electron microscopy)

| | | LOCATION 6 | LOCATION 5 | LOCATION 4 | LOCATION 3 | LOCATION 2 | LOCATION 1 |
|---|---|---|---|---|---|---|---|
| | BEFORE TREATMENT (hair width μm) | | | | | | |
| | | 86.6 | 88.0 | 76.0 | 67.2 | 47.1 | 61.2 |
| | | 90.0 | 88.0 | 69.8 | 66.2 | 55.7 | 71.8 |
| | | 85.4 | | 72.3 | 54.6 | 51.4 | 69.3 |
| AVERAGE | 71.6 | 87.3 | 88.0 | 72.7 | 62.7 | 51.4 | 67.4 |
| SD | 2.6 | 1.7 | 0.0 | 2.2 | 5.0 | 3.0 | 3.9 |
| | AFTER TREATMENT (hair width μm) | | | | | | |
| | | 106.3 | 112.6 | 91.3 | 72.3 | 76.6 | 108.0 |
| | | 97.3 | 104.1 | 88.7 | 72.6 | 89.5 | 105.7 |
| | | 82.7 | 102.6 | 73.5 | 90.7 | 74.5 | |
| AVERAGE | 92.0 | 95.4 | 106.4 | 84.5 | 78.5 | 80.2 | 106.9 |
| SD | 5.5 | 8.4 | 3.8 | 6.8 | 7.5 | 5.7 | 0.9 |
| t TEST | 5.47E−07 | | | | | | |

### EXAMPLE 9

Synergy: In addition to evaluating the effects of the mixtures of the current invention, the effects of each individual herb comprising the mixtures were assessed as well in order to assess synergistic effects. Eight subjects, Female #3-10, aged 24-47, who suffer from thin, flat hair or hair loss were treated for a period of three weeks, either with a rosemary extract (Female #3), thyme extract (Female #4), nettle extract (Female #5), mamaki extract (Female #6), sage extract (Female #7), spearmint extract (Female #8), a mixture comprising thyme, rosemary and nettle (‘Mix 1’, Female #9) or a mixture comprising sage, mamaki and spearmint (‘Mix 2’, Female #10). Subjects washed their hair with the boiled extracts at least twice a week, and hairs from the frontal area of the scalp were sampled and analyzed in two different time points during the treatment period:
a. Before the treatment—hairs from the frontal area of the scalp of each subject were collected and the hairs' width was measured under a microscope.
b. After 3 weeks of using either the rosemary extract, thyme extract, nettle extract, mamaki extract, sage extract, spearmint extract or the mixtures of the current invention ({rosemary+nettle+thyme} or {sage+spearmint+mamaki}), hairs from the frontal area of the scalp of each subject were collected and the hairs' width was measured under a microscope.

The results show a significant increase in the hair's width only after using the mixtures of the current invention (see Table 5). The average hair's width increased by 62.62% for mixture 1 and by 41% for mixture 2, compared to untreated hair, whereas for thyme, sage, rosemary, mint, nettle and mamaki extracts the average increase was 1.03%, 0.67%, 6.93%, 5.15%, 10.86% and 4.13% respectively. Furthermore, the relative improvements observed after using the rosemary and nettle extracts were significant, but not as statistically significant as the results obtained after using the mixtures. These results confirm that the mixtures of the current invention have synergistic effects compared to using each herb separately, as their efficacy surpasses the total summing of the individual treatments.

TABLE 5
Hair width before and after treatment with each herb separately and the
mixtures of the current invention (as determined by electron microscopy).

| | BEFORE | | | | | | | | |
|---|---|---|---|---|---|---|---|---|---|
| | TREATMENT | | | | | | | | |
| | (hair width | AFTER TREATMENT (hair width μm) | | | | | | | |
| | μm) | thyme | % | sage | % | rosemary | % | mint | % |
| sample 1 | 61.11 | 63.9 | | 61.22 | | 68.14 | | 63.98 | |
| sample 2 | 64.6 | 65.7 | | 63.55 | | 69.36 | | 72.56 | |
| sample 3 | 73.08 | 62.9 | | 62.78 | | 70.12 | | 66.34 | |
| sample 4 | 63.59 | 67.89 | | 71.89 | | 72.34 | | 68.87 | |
| sample 5 | 62.31 | 70.2 | | 65.89 | | 69.78 | | 64.57 | |
| sample 6 | 65.37 | 63.5 | | 67.41 | | 67.37 | | 73.85 | |
| AVG | 65.01 | 65.68 | 1.03 | 65.45 | 0.67 | 69.52 | 6.93 | 68.36 | 5.15 |
| SD | 4.24 | 2.86 | | 3.85 | | 1.73 | | 4.139 | |
| t-TEST | | 0.75 | | 0.85 | | 0.04 | | 0.196 | |

| | AFTER TREATMENT (hair width μm) | | | | | | | |
|---|---|---|---|---|---|---|---|---|
| | nettle | % | mamaki | % | MIX 1 | % | MIX 2 | % |
| sample 1 | 79.58 | | 62.39 | | 112.65 | | 94.35 | |
| sample 2 | 74.32 | | 64.75 | | 104.18 | | 92.87 | |
| sample 3 | 72.68 | | 61.85 | | 102.64 | | 85.63 | |
| sample 4 | 65.48 | | 74.59 | | 106.47 | | 89.42 | |
| sample 5 | 70.92 | | 72.84 | | 109.82 | | 90.63 | |
| sample 6 | 69.45 | | 69.76 | | 98.56 | | 96.95 | |
| AVG | 72.07 | 10.86 | 67.70 | 4.13 | 105.72 | 62.62 | 91.64 | 41 |
| SD | 4.76 | | 5.46 | | 5.06 | | 3.98 | |
| t-TEST | 0.02 | | 0.36 | | 3.3E−08 | | 5.5E−07 | |

AVG = average, SD = standard deviation, the % represent the average increase in the hair's width.
MIX 1 = a mixture comprising rosemary, thyme and nettle.
MIX 2 = a mixture comprising sage, spearmint and mamaki.

1. A synergic water based herbal mixture for the use in the treatment of scalp and hair-disorders of a mammalian subject, comprising at least three herbs, wherein first herb is at least one of Urtica diocia or Pipturus albidus, having premixing specific rotation value (PSRV) [α]_{D}²⁰=−99 and GCMS 6 peaks within RTs 8.23-8.82 min., second herb is at least one of Thymus vulgaris or Salvia officinalis having a PSRV [α]_{D}²⁰=+33 and RTs 12.67, 12.84, 13.14 and 18.14 min., and third herb is at least one of Rosmarinus officinalis or Mentha viridis having a PSRV [α]_{D}²⁰=−5 and RTs 11.52, 11.59, 15.53, 17.21 and 18.15 min; wherein said mixture is characterized by post mixture specific rotation value [α]_{D}²⁰=+18 and by GCMS first, second and third peaks of retention time 11.55, 11.94 and 12.69 min., respectively; wherein said mixture is characterized by said first peak (11.55 min) to said second peak (11.94 min) ratio between 1:7 to 1:2; said second peak to said third peak (12.69 min.) ratio between 1:4 to 3:2; and said first peak to said third peak ratio of between 1:12 to 1:3 as determined by GCMS peak areas; and further wherein said GCMS is Model GCD 1800, utilized under the following conditions: preparation for analysis was performed by basic extraction (pH=10) in dichloromethane, 3 times, ratio of 1 to 5 sample to solvent; acid extraction (pH=3) in dichloromethane, 3 times, ratio of 1 to 5 sample to solvent; consolidation of the extracts and evaporating in vacuum to a volume of 10 ml; injection of 2 microliters of the sample into the GCMS, using the following program: 60 degrees C. for 7 minutes, increasing at 15 degrees C. per minute to 320 degrees C., staying at 320 degrees C. for 15 minutes.

2. The mixture of claim 1, wherein first peak area of first peak to second peak to third peak ratio is from 5:17:45 and 5:8:18.

3. The mixture of claim 1, wherein said mixture comprises at least one compound selected from the group consisting of water, ethanol, anti-bacterial agents, moisturizers, hydration agents, penetration agents, preservatives, viscosity-controlling agents anesthetics, vitamins, anti-itch actives, botanical extracts, color agents, enzymes, conditioning agents, darkening or lightening agents, nutrients, micronutrients, fragrances, glitters, hair pigment additives, humectants, mica, minerals, oils, polyphenols, silicones or derivatives thereof, sun-blocks, surfactants, vitamins, waxes, solvents, rheology modifiers, suspending agent, thickeners, hair health actives, anti-dandruff mixtures, anti-oxidants, pigments, abrasives, absorbents, biological actives, buffering agents, chelating agents, opacifying agents, pH adjusters, chelates, perfumes, brighteners, sensates, attractants, dyes, bleaches and any combination thereof.

4. The mixture of claim 1, wherein said mixture is provided in a form selected from a group consisting of liquid, solution, capsule, gel, dissolving capsule, capsule comprising a filter, infusion, cream, ointment, electroporation formulation, foam, gel, suspension, emulsion, lotion, microspheres, microbeads, mud, oil, paste, ointment, patch, powder, roller stick, soap, surfactant, spray, shampoo conditioner, a rinse-off conditioner, a tablet, a pill or a granulated powder, nutraceutical, a functional food, a dietary supplement, a food supplement, a pharmaceutical product or a cosmetic product topical, subcutaneous, dermal, oral, and any combination thereof.

5. The mixture according to claim 1, wherein said treatment comprises: applying said mixture on said mammalian head, and massaging said mixture in scalp of said mammalian subject.

6. The mixture according to claim 1, wherein said mammalian subject is selected from a group consisting of humans, monkeys, cats, cows, dogs, gerbils, goats, guinea pigs, hamsters, horses, mice, prairie dogs, rabbits, rats, sheep and squirrels.

7. The mixture according to claim 1, wherein said treatment is selected from a group consisting of inducing new hair growth, stimulating new hair growth, increasing hair growth, preventing hair regression, cleansing the hair, cleansing the scalp, beautifying the hair, promoting attraction, altering appearance of hair, and any combination thereof.

8. The mixture according to claim 1, wherein said mixture is produced by a step of admixing said mixtures with at least one member of a group consisting of water, ethanol, one or more anti-bacterial agents, moisturizers and hydration agents, penetration agents, preservatives, viscosity-controlling agents anesthetics, vitamins, anti-itch actives, botanical extracts, color agents, enzymes, conditioning agents, darkening or lightening agents, fragrances, glitters, hair pigment additives, humectants, mica, minerals, oils, polyphenols, silicones or derivatives thereof, sun-blockers, surfactants, vitamins, micronutrients, nutrients, waxes, solvents, rheology modifiers, suspending agents, thickeners, hair health actives, anti-dandruff actives, anti-oxidants, pigments, abrasives, absorbents, biological actives, buffering agents, chelating agents, opacifying agents, pH adjusters, chelates, perfumes, brighteners, sensates, attractants, dyes, bleaches and any combination thereof.

9. The mixture according to claim 1, wherein said mixture is further produced, in the form selected from a group consisting of liquid, solution, capsule, gel, dissolving capsule, capsule comprising of filter, infusion, cream, ointment, electroporation formulation, emulsion, foam, gel, suspension, emulsion, lotion, microspheres, microbeads, mud, oil, paste, ointment, patch, powder, roller stick, soap, surfactant, spray, shampoo conditioner, a rinse-off conditioner, and any combination thereof.

10. The mixture according to claim 1, wherein said scalp and hair-disorders of a mammalian subject are selected from the group consisting of alopecia, accidental injury, damage to hair follicles, surgical trauma, burn wound, radiation or chemotherapy treatment site, incisional wound, donor site wound from skin transplant and ulcer; a desire to modify physical appearance, and any combination thereof; or to enhance one or more hair properties selected from the group consisting of brilliance, fullness, gloss, glow, length, luster, patina, sheen, shine, thickness, volume and any combination thereof.

11. A kit for providing a mixture for use in the treatment of hair or scalp disorders in a mammalian subject , inducing or stimulating new hair growth, increasing hair growth or preventing hair regression in a mammalian subject, cleansing the hair, cleansing the scalp, beautifying the hair, promoting attraction and altering the appearance of hair and any combination thereof said kit comprising: a synergic water based herbal mixture comprising at least three herbs, wherein first herb is at least one of Urtica diocia or Pipturus albidus having premixing specific rotation value (PSRV) [α]_{D}²⁰=−99 and GCMS 6 peaks within RTs 8.23-8.82 min., second herb is at least one of Thymus vulgaris or Salvia officinalis having a PSRV [α]_{D}²⁰=+33 and RTs 12.67, 12.84, 13.14 and 18.14 min., and third herb is at least one of Rosmarinus officinalis or Mentha viridis having a PSRV [α]_{D}²⁰=−5 and RTs 11.52, 11.59, 15.53, 17.21 and 18.15 min; wherein said mixture is characterized by post mixture specific rotation value [α]_{D}²⁰=+18 and by GCMS first, second and third peaks of retention time 11.55, 11.94 and 12.69 min., respectively; wherein said mixture is characterized by said first peak (11.55 min) to said second peak (11.94 min) ratio between 1:7 to 1:2; said second peak to said third peak (12.69 min.) ratio between 1:4 to 3:2; and said first peak to said third peak ratio of between 1:12 to 1:3 as determined by GCMS peak areas; and further wherein said GCMS is Model GCD 1800, utilized under the following conditions: preparation for analysis was performed by basic extraction (pH=10) in dichloromethane, 3 times, ratio of 1 to 5 sample to solvent; acid extraction (pH=3) in dichloromethane, 3 times, ratio of 1 to 5 sample to solvent; consolidation of the extracts and evaporating in vacuum to a volume of 10 ml; injection of 2 microliters of the sample into the GCMS, using the following program: 60 degrees C. for 7 minutes, increasing at 15 degrees C. per minute to 320 degrees C., staying at 320 degrees C. for 15 minutes.

12. The kit of claim 11, wherein peak area of first peak to second peak to third peak ratio is from 5:17:45 to 5:8:18.

13. The kit of claim 11, wherein said application means is selected from the group consisting of, brushes, combs, controlled-release matrices, fabrics, pumps, sprayers, especially aerosol spray dispensers, self-pressurized spray dispensers and non-aerosol spray dispensers, sachets, tea bags, paper tea bags, corn-made tea bags, fabric tea bags, pouches, bags, aluminum bags, cloth bags, plastic bags, filtered bottles, bottles with stainless steel filters, bottles with plastic filters, stainless steel filters, plastic filters, droppers, capsules, balls, compressed cubes, filter, corn paper filters, a filter cloths, organic filters, paper filters, ampoules, pouches, flasks, rollers, plastic rollers, plastic rollers and stainless steel, wands, or a container selected from the group consisting of bottles, cans, tubes, vials, dispensers, sachets, tea bags, paper tea bags, corn-made tea bags, fabric tea bags, pouches, bags, aluminum bags, cloth bags, plastic bags, filtered bottles, bottles with stainless steel filters, bottles with plastic filters, and any combination thereof.

14. The kit of claim 11, wherein said mixture further comprises at least one member of a group consisting of water, ethanol, anti-bacterial agents, moisturizers and hydration agents, penetration agents, preservatives, viscosity-controlling agents anesthetics, vitamins, anti-itch actives, botanical extracts, color agents, enzymes, conditioning agents, darkening or lightening agents, nutrient, micronutrient, fragrance, glitter, hair pigment additives, humectants, mica, minerals, oils, polyphenols, silicones or derivatives thereof, sun-blocks, surfactants, vitamins, waxes, solvents, rheology modifiers, suspending agent, thickeners, hair health actives, anti-dandruff actives, anti-oxidants, pigments, abrasives, absorbents, biological actives, buffering agents, chelating agents, opacifying agents, pH adjusters, chelates, perfumes, brighteners, sensates, attractants, dyes, bleaches and any combination thereof.

15. The kit of claim 11, wherein said mixture is provided in the form selected from a group consisting of liquid, solution, capsule, gel, dissolving capsule, capsule comprising of filter, infusion, cream, ointment, electroporation formulation, emulsion, foam, gel, suspension, emulsion, lotion, microspheres, microbeads, mud, oil, paste, ointment, patch, powder, roller stick, soap, surfactant, spray, shampoo conditioner, a rinse-off conditioner, and any combination thereof.

16. The kit of claim 11, wherein said mixture is configured to be administrable in a manner selected from a group consisting of topical, subcutaneous, dermal, oral, tablet, a pill or a granulated powder, nutraceutical, a functional food, a dietary supplement, a food supplement, a pharmaceutical product or a cosmetic product and any combination thereof.

17. The kit of claim 11, wherein said hair or scalp disorders are selected from the group consisting of alopecia, accidental injury, damage to hair follicles, surgical trauma, burn wound, radiation or chemotherapy treatment site, incisional wound, donor site wound from skin transplant and ulcer; a desire to modify physical appearance, or any combination thereof.

18. The kit of claim 11, wherein said mixture configured to enhance one or more hair properties selected from the group consisting of brilliance, fullness, gloss, glow, length, luster, patina, sheen, shine, thickness, volume and any combination thereof.

19. A method for preparation of a synergic water based herbal mixture for scalp or hair treatment, wherein said method comprises steps of: mixing at least three herbs, wherein first herb is at least one of Urtica diocia or Pipturus albidus having premixing specific rotation value (PSRV) [α]_{D}²⁰=−99 and GCMS 6 peaks within RTs 8.23-8.82 min., second herb is at least one of Thymus vulgaris or Salvia officinalis having a PSRV [α]_{D}²⁰=+33 and RTs 12.67, 12.84, 13.14 and 18.14 min., and third herb is at least one of Rosmarinus officinalis or Mentha viridis having a PSRV [α]_{D}²⁰=−5 and RTs 11.52, 11.59, 15.53, 17.21 and 18.15 min.; thereby producing a mixture; and immersing said mixture in boiling water for 10 to 180 minutes; wherein said mixture is characterized by post mixture specific rotation value [α]_{D}²⁰=+18 and by GCMS first, second and third peaks of retention time 11.55, 11.94 and 12.69 min., wherein said mixture is characterized by said first peak (11.55 min) to said second peak (11.94 min) ratio between 1:7 to 1:2; said second peak to said third peak (12.69 min.) ratio between 1:4 to 3:2; and said first peak to said third peak ratio of between 1:12 to 1:3 as determined by GC peak areas; and further wherein utilizing GCMS, Model GCD 1800 under the following conditions: preparation for analysis was performed by basic extraction (pH=10) in dichloromethane, 3 times, ratio of 1 to 5 sample to solvent; acid extraction (pH=3) in dichloromethane, 3 times, ratio of 1 to 5 sample to solvent; consolidation of the extracts and evaporating in vacuum to a volume of 10 ml; injection of 2 microliters of the sample into the GCMS, using the following program: 60 degrees C. for 7 minutes, increasing at 15 C per minute to 320 degrees C., staying at 320 degrees C. for 15 minutes.

20. The method according to claim 19, wherein said step of immersing is followed by boiling said mixture and then cooking the boiled mixture for additional up to 60 minutes in 40° C. to 80° C.
